SEQUENCE LISTING (6 sequences)

SEQ ID NO: 1 - DNA, 102 bp, adeno-associated virus serotype 2
cgacattttgcgacaccatgtggtcacgctgggtatttaagcccgagtgagcacgcagggtctccattttgaagcgggaggtttgaacgcgcagccgccatg

SEQ ID NO: 2 - DNA, 4683 bp, adeno-associated virus serotype 6
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagcgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgacttcctggtccagtggcgccgcgtgagtaaggccccggaggccctcttctttgttcagttcgagaagggcgagtcctacttccacctccatattctggtggagaccacgggggtcaaatccatggtgctgggccgcttcctgagtcagattagggacaagctggtgcagaccatctaccgcgggatcgagccgaccctgcccaactggttcgcggtgaccaagacgcgtaatggcgccggaggggggaacaaggtggtggacgagtgctacatccccaactacctcctgcccaagactcagcccgagctgcagtgggcgtggactaacatggaggagtatataagcgcgtgtttaaacctggccgagcgcaaacggctcgtggcgcacgacctgacccacgtcagccagacccaggagcagaacaaggagaatctgaaccccaattctgacgcgcctgtcatccggtcaaaaacctccgcacgctacatggagctggtcgggtggctggtggaccggggcatcacctccgagaagcagtggatccaggaggaccaggcctcgtacatctccttcaacgccgcctccaactcgcggtcccagatcaaggccgctctggacaatgccggcaagatcatggcgctgaccaaatccgcgcccgactacctggtaggccccgctccgcccgccgacattaaaaccaaccgcatttaccgcatcctggagctgaacggctacgaccctgcctacgccggctccgtctttctcggctgggcccagaaaaggttcggaaaacgcaacaccatctggctgtttgggccggccaccacgggcaagaccaacatcgcggaagccatcgcccacgccgtgcccttctacggctgcgtcaactggaccaatgagaactttcccttcaacgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgatcccacccccgtgatcgtcacctccaacaccaacatgtgcgccgtgattgacgggaacagcaccaccttcgagcaccagcagccgttgcaggaccggatgttcaaatttgaactcacccgccgtctggagcatgactttggcaaggtgacaaagcaggaagtcaaagagttcttccgctgggcgcaggatcacgtgaccgaggtggcgcatgagttctacgtcagaaagggtggagccaacaagagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaaaacatgcgagagaatgaatcagaatttcaacatttgcttcacgcacgggaccagagactgttcagaatgtttccccggcgtgtcagaatctcaaccggtcgtcagaaagaggacgtatcggaaactctgtgccattcatcatctgctggggcgggctcccgagattgcttgctcggcctgcgatctggtcaacgtggatctggatgactgtgtttctgagcaataaatgacttaaaccaggtatggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaattgtgtgttaatcaataaaccggttaattcgtgtcagttgaactttggtctcatgtcgttattatcttatctggtcaccatagcaaccggttacacattaactgcttagttgcgcttcgcgaatacccctagtgatggagttgcccactccctctatgcgcgctcgctcgctcggtggggccggcagagcagagctctgccgtctgcggacctttggtccgcaggccccaccgagcgagcgagcgcgcatagagggagtgggcaa

SEQ ID NO: 3 - DNA, 4718 bp, adeno-associated virus serotype 1
ttgcccactccctctctgcgcgctcgctcgctcggtggggcctgcggaccaaaggtccgcagacggcagagctctgctctgccggccccaccgagcgagcgagcgcgcagagagggagtgggcaactccatcactaggggtaatcgcgaagcgcctcccacgctgccgcgtcagcgctgacgtaaattacgtcataggggagtggtcctgtattagctgtcacgtgagtgcttttgcgacattttgcgacaccacgtggccatttagggtatatatggccgagtgagcgagcaggatctccattttgaccgcgaaatttgaacgagcagcagccatgccgggcttctacgagatcgtgatcaaggtgccgagcgacctggacgagcacctgccgggcatttctgactcgtttgtgagctgggtggccgagaaggaatgggagctgcccccggattctgacatggatctgaatctgattgagcaggcacccctgaccgtggccgagaagctgcagcgcgacttcctggtccaatggcgccgcgtgagtaaggccccggaggccctcttctttgttcagttcgagaagggcgagtcctacttccacctccatattctggtggagaccacgggggtcaaatccatggtgctgggccgcttcctgagtcagattagggacaagctggtgcagaccatctaccgcgggatcgagccgaccctgcccaactggttcgcggtgaccaagacgcgtaatggcgccggaggggggaacaaggtggtggacgagtgctacatccccaactacctcctgcccaagactcagcccgagctgcagtgggcgtggactaacatggaggagtatataagcgcctgtttgaacctggccgagcgcaaacggctcgtggcgcagcacctgacccacgtcagccagacccaggagcagaacaaggagaatctgaaccccaattctgacgcgcctgtcatccggtcaaaaacctccgcgcgctacatggagctggtcgggtggctggtggaccggggcatcacctccgagaagcagtggatccaggaggaccaggcctcgtacatctccttcaacgccgcttccaactcgcggtcccagatcaaggccgctctggacaatgccggcaagatcatggcgctgaccaaatccgcgcccgactacctggtaggccccgctccgcccgcggacattaaaaccaaccgcatctaccgcatcctggagctgaacggctacgaacctgcctacgccggctccgtctttctcggctgggcccagaaaaggttcgggaagcgcaacaccatctggctgtttgggccggccaccacgggcaagaccaacatcgcggaagccatcgcccacgccgtgcccttctacggctgcgtcaactggaccaatgagaactttcccttcaatgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgaccccacccccgtgatcgtcacctccaacaccaacatgtgcgccgtgattgacgggaacagcaccaccttcgagcaccagcagccgttgcaggaccggatgttcaaatttgaactcacccgccgtctggagcatgactttggcaaggtgacaaagcaggaagtcaaagagttcttccgctgggcgcaggatcacgtgaccgaggtggcgcatgagttctacgtcagaaagggtggagccaacaaaagacccgcccccgatgacgcggataaaagcgagcccaagcgggcctgcccctcagtcgcggatccatcgacgtcagacgcggaaggagctccggtggactttgccgacaggtaccaaaacaaatgttctcgtcacgcgggcatgcttcagatgctgtttccctgcaagacatgcgagagaatgaatcagaatttcaacatttgcttcacgcacgggacgagagactgttcagagtgcttccccggcgtgtcagaatctcaaccggtcgtcagaaagaggacgtatcggaaactctgtgccattcatcatctgctggggcgggctcccgagattgcttgctcggcctgcgatctggtcaacgtggacctggatgactgtgtttctgagcaataaatgacttaaaccaggtatggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaattacgtgttaatcaataaaccggttgattcgtttcagttgaactttggtctcctgtccttcttatcttatcggttaccatggttatagcttacacattaactgcttggttgcgcttcgcgataaaagacttacgtcatcgggttacccctagtgatggagttgcccactccctctctgcgcgctcgctcgctcggtggggcctgcggaccaaaggtccgcagacggcagagctctgctctgccggccccaccgagcgagcgagcgcgcagagagggagtgggcaa

SEQ ID NO: 4 - DNA, 4726 bp, adeno-associated virus serotype 3
ttggccactccctctatgcgcactcgctcgctcggtggggcctggcgaccaaaggtcgccagacggacgtgctttgcacgtccggccccaccgagcgagcgagtgcgcatagagggagtggccaactccatcactagaggtatggcagtgacgtaacgcgaagcgcgcgaagcgagaccacgcctaccagctgcgtcagcagtcaggtgacccttttgcgacagtttgcgacaccacgtggccgctgagggtatatattctcgagtgagcgaaccaggagctccattttgaccgcgaaatttgaacgagcagcagccatgccggggttctacgagattgtcctgaaggtcccgagtgacctggacgagcgcctgccgggcatttctaactcgtttgttaactgggtggccgagaaggaatgggacgtgccgccggattctgacatggatccgaatctgattgagcaggcacccctgaccgtggccgaaaagcttcagcgcgagttcctggtggagtggcgccgcgtgagtaaggccccggaggccctcttttttgtccagttcgaaaagggggagacctacttccacctgcacgtgctgattgagaccatcggggtcaaatccatggtggtcggccgctacgtgagccagattaaagagaagctggtgacccgcatctaccgcggggtcgagccgcagcttccgaactggttcgcggtgaccaaaacgcgaaatggcgccgggggcgggaacaaggtggtggacgactgctacatccccaactacctgctccccaagacccagcccgagctccagtgggcgtggactaacatggaccagtatttaagcgcctgtttgaatctcgcggagcgtaaacggctggtggcgcagcatctgacgcacgtgtcgcagacgcaggagcagaacaaagagaatcagaaccccaattctgacgcgccggtcatcaggtcaaaaacctcagccaggtacatggagctggtcgggtggctggtggaccgcgggatcacgtcagaaaagcaatggattcaggaggaccaggcctcgtacatctccttcaacgccgcctccaactcgcggtcccagatcaaggccgcgctggacaatgcctccaagatcatgagcctgacaaagacggctccggactacctggtgggcagcaacccgccggaggacattaccaaaaatcggatctaccaaatcctggagctgaacgggtacgatccgcagtacgcggcctccgtcttcctgggctgggcgcaaaagaagttcgggaagaggaacaccatctggctctttgggccggccacgacgggtaaaaccaacatcgcggaagccatcgcccacgccgtgcccttctacggctgcgtaaactggaccaatgagaactttcccttcaacgattgcgtcgacaagatggtgatctggtgggaggagggcaagatgacggccaaggtcgtggagagcgccaaggccattctgggcggaagcaaggtgcgcgtggaccaaaagtgcaagtcatcggcccagatcgaacccactcccgtgatcgtcacctccaacaccaacatgtgcgccgtgattgacgggaacagcaccaccttcgagcatcagcagccgctgcaggaccggatgtttgaatttgaacttacccgccgtttggaccatgactttgggaaggtcaccaaacaggaagtaaaggactttttccggtgggcttccgatcacgtgactgacgtggctcatgagttctacgtcagaaagggtggagctaagaaacgccccgcctccaatgacgcggatgtaagcgagccaaaacgggagtgcacgtcacttgcgcagccgacaacgtcagacgcggaagcaccggcggactacgcggacaggtaccaaaacaaatgttctcgtcacgtgggcatgaatctgatgctttttccctgtaaaacatgcgagagaatgaatcaaatttccaatgtctgttttacgcatggtcaaagagactgtggggaatgcttccctggaatgtcagaatctcaacccgtttctgtcgtcaaaaagaagacttatcagaaactgtgtccaattcatcatatcctgggaagggcacccgagattgcctgttcggcctgcgatttggccaatgtggacttggatgactgtgtttctgagcaataaatgacttaaaccaggtatggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagggggctgtagatcagtctcctcaggaaccggactcatcatctggtgttggcaaatcgggcaaacagcctgccagaaaaagactaaatttcggtcagactggagactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttagaggggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactggaactgtcaatcatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtgaatcctggttaatcaataaaccgtttaattcgtttcagttgaactttggctcttgtgcacttctttatctttatcttgtttccatggctactgcgtagataagcagcggcctgcggcgcttgcgcttcgcggtttacaactgctggttaatatttaactctcgccatacctctagtgatggagttggccactccctctatgcgcactcgctcgctcggtggggcctggcgaccaaaggtcgccagacggacgtgctttgcacgtccggccccaccgagcgagcgagtgcgcatagagggagtggccaa

SEQ ID NO: 5 - DNA, 4767 bp, adeno-associated virus serotype 4
ttggccactccctctatgcgcgctcgctcactcactcggccctggagaccaaaggtctccagactgccggcctctggccggcagggccgagtgagtgagcgagcgcgcatagagggagtggccaactccatcatctaggtttgcccactgacgtcaatgtgacgtcctagggttagggaggtccctgtattagcagtcacgtgagtgtcgtatttcgcggagcgtagcggagcgcataccaagctgccacgtcacagccacgtggtccgtttgcgacagtttgcgacaccatgtggtcaggagggtatataaccgcgagtgagccagcgaggagctccattttgcccgcgaattttgaacgagcagcagccatgccggggttctacgagatcgtgctgaaggtgcccagcgacctggacgagcacctgcccggcatttctgactcttttgtgagctgggtggccgagaaggaatgggagctgccgccggattctgacatggacttgaatctgattgagcaggcacccctgaccgtggccgaaaagctgcaacgcgagttcctggtcgagtggcgccgcgtgagtaaggccccggaggccctcttctttgtccagttcgagaagggggacagctacttccacctgcacatcctggtggagaccgtgggcgtcaaatccatggtggtgggccgctacgtgagccagattaaagagaagctggtgacccgcatctaccgcggggtcgagccgcagcttccgaactggttcgcggtgaccaagacgcgtaatggcgccggaggcgggaacaaggtggtggacgactgctacatccccaactacctgctccccaagacccagcccgagctccagtgggcgtggactaacatggaccagtatataagcgcctgtttgaatctcgcggagcgtaaacggctggtggcgcagcatctgacgcacgtgtcgcagacgcaggagcagaacaaggaaaaccagaaccccaattctgacgcgccggtcatcaggtcaaaaacctccgccaggtacatggagctggtcgggtggctggtggaccgcgggatcacgtcagaaaagcaatggatccaggaggaccaggcgtcctacatctccttcaacgccgcctccaactcgcggtcacaaatcaaggccgcgctggacaatgcctccaaaatcatgagcctgacaaagacggctccggactacctggtgggccagaacccgccggaggacatttccagcaaccgcatctaccgaatcctcgagatgaacgggtacgatccgcagtacgcggcctccgtcttcctgggctgggcgcaaaagaagttcgggaagaggaacaccatctggctctttgggccggccacgacgggtaaaaccaacatcgcggaagccatcgcccacgccgtgcccttctacggctgcgtgaactggaccaatgagaactttccgttcaacgattgcgtcgacaagatggtgatctggtgggaggagggcaagatgacggccaaggtcgtagagagcgccaaggccatcctgggcggaagcaaggtgcgcgtggaccaaaagtgcaagtcatcggcccagatcgacccaactcccgtgatcgtcacctccaacaccaacatgtgcgcggtcatcgacggaaactcgaccaccttcgagcaccaacaaccactccaggaccggatgttcaagttcgagctcaccaagcgcctggagcacgactttggcaaggtcaccaagcaggaagtcaaagactttttccggtgggcgtcagatcacgtgaccgaggtgactcacgagttttacgtcagaaagggtggagctagaaagaggcccgcccccaatgacgcagatataagtgagcccaagcgggcctgtccgtcagttgcgcagccatcgacgtcagacgcggaagctccggtggactacgcggacaggtaccaaaacaaatgttctcgtcacgtgggtatgaatctgatgctttttccctgccggcaatgcgagagaatgaatcagaatgtggacatttgcttcacgcacggggtcatggactgtgccgagtgcttccccgtgtcagaatctcaacccgtgtctgtcgtcagaaagcggacgtatcagaaactgtgtccgattcatcacatcatggggagggcgcccgaggtggcctgctcggcctgcgaactggccaatgtggacttggatgactgtgacatggaacaataaatgactcaaaccagatatgactgacggttaccttccagattggctagaggacaacctctctgaaggcgttcgagagtggtgggcgctgcaacctggagcccctaaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctcaaggccggtgacaacccctacctcaagtacaaccacgccgacgcggagttccagcagcggcttcagggcgacacatcgtttgggggcaacctcggcagagcagtcttccaggccaaaaagagggttcttgaacctcttggtctggttgagcaagcgggtgagacggctcctggaaagaagagaccgttgattgaatccccccagcagcccgactcctccacgggtatcggcaaaaaaggcaagcagccggctaaaaagaagctcgttttcgaagacgaaactggagcaggcgacggaccccctgagggatcaacttccggagccatgtctgatgacagtgagatgcgtgcagcagctggcggagctgcagtcgagggcggacaaggtgccgatggagtgggtaatgcctcgggtgattggcattgcgattccacctggtctgagggccacgtcacgaccaccagcaccagaacctgggtcttgcccacctacaacaaccacctctacaagcgactcggagagagcctgcagtccaacacctacaacggattctccaccccctggggatactttgacttcaaccgcttccactgccacttctcaccacgtgactggcagcgactcatcaacaacaactggggcatgcgacccaaagccatgcgggtcaaaatcttcaacatccaggtcaaggaggtcacgacgtcgaacggcgagacaacggtggctaataaccttaccagcacggttcagatctttgcggactcgtcgtacgaactgccgtacgtgatggatgcgggtcaagagggcagcctgcctccttttcccaacgacgtctttatggtgccccagtacggctactgtggactggtgaccggcaacacttcgcagcaacagactgacagaaatgccttctactgcctggagtactttccttcgcagatgctgcggactggcaacaactttgaaattacgtacagttttgagaaggtgcctttccactcgatgtacgcgcacagccagagcctggaccggctgatgaaccctctcatcgaccagtacctgtggggactgcaatcgaccaccaccggaaccaccctgaatgccgggactgccaccaccaactttaccaagctgcggcctaccaacttttccaactttaaaaagaactggctgcccgggccttcaatcaagcagcagggcttctcaaagactgccaatcaaaactacaagatccctgccaccgggtcagacagtctcatcaaatacgagacgcacagcactctggacggaagatggagtgccctgacccccggacctccaatggccacggctggacctgcggacagcaagttcagcaacagccagctcatctttgcggggcctaaacagaacggcaacacggccaccgtacccgggactctgatcttcacctctgaggaggagctggcagccaccaacgccaccgatacggacatgtggggcaacctacctggcggtgaccagagcaacagcaacctgccgaccgtggacagactgacagccttgggagccgtgcctggaatggtctggcaaaacagagacatttactaccagggtcccatttgggccaagattcctcataccgatggacactttcacccctcaccgctgattggtgggtttgggctgaaacacccgcctcctcaaatttttatcaagaacaccccggtacctgcgaatcctgcaacgaccttcagctctactccggtaaactccttcattactcagtacagcactggccaggtgtcggtgcagattgactgggagatccagaaggagcggtccaaacgctggaaccccgaggtccagtttacctccaactacggacagcaaaactctctgttgtgggctcccgatgcggctgggaaatacactgagcctagggctatcggtacccgctacctcacccaccacctgtaataacctgttaatcaataaaccggtttattcgtttcagttgaactttggtctccgtgtccttcttatcttatctcgtttccatggctactgcgtacataagcagcggcctgcggcgcttgcgcttcgcggtttacaactgccggttaatcagtaacttctggcaaaccagatgatggagttggccacattagctatgcgcgctcgctcactcactcggccctggagaccaaaggtctccagactgccggcctctggccggcagggccgagtgagtgagcgagcgcgcatagagggagtggccaa

SEQ ID NO: 6 - DNA, 4404 bp, adeno-associated virus serotype 5
cgcgacaggggggagagtgccacactctcaagcaagggggttttgtaagcagtgatgtcataatgatgtaatgcttattgtcacgcgatagttaatgattaacagtcatgtgatgtgttttatccaataggaagaaagcgcgcgtatgagttctcgcgagacttccggggtataaaagaccgagtgaacgagcccgccgccattctttgctctggactgctagaggaccctcgctgccatggctaccttctatgaagtcattgttcgcgtcccatttgacgtggaggaacatctgcctggaatttctgacagctttgtggactgggtaactggtcaaatttgggagctgcctccagagtcagatttaaatttgactctggttgaacagcctcagttgacggtggctgatagaattcgccgcgtgttcctgtacgagtggaacaaattttccaagcaggagtccaaattctttgtgcagtttgaaaagggatctgaatattttcatctgcacacgcttgtggagacctccggcatctcttccatggtcctcggccgctacgtgagtcagattcgcgcccagctggtgaaagtggtcttccagggaattgaaccccagatcaacgactgggtcgccatcaccaaggtaaagaagggcggagccaataaggtggtggattctgggtatattcccgcctacctgctgccgaaggtccaaccggagcttcagtgggcgtggacaaacctggacgagtataaattggccgccctgaatctggaggagcgcaaacggctcgtcgcgcagtttctggcagaatcctcgcagcgctcgcaggaggcggcttcgcagcgtgagttctcggctgacccggtcatcaaaagcaagacttcccagaaatacatggcgctcgtcaactggctcgtggagcacggcatcacttccgagaagcagtggatccaggaaaatcaggagagctacctctccttcaactccaccggcaactctcggagccagatcaaggccgcgctcgacaacgcgaccaaaattatgagtctgacaaaaagcgcggtggactacctcgtggggagctccgttcccgaggacatttcaaaaaacagaatctggcaaatttttgagatgaatggctacgacccggcctacgcgggatccatcctctacggctggtgtcagcgctccttcaacaagaggaacaccgtctggctctacggacccgccacgaccggcaagaccaacatcgcggaggccatcgcccacactgtgcccttttacggctgcgtgaactggaccaatgaaaactttccctttaatgactgtgtggacaaaatgctcatttggtgggaggagggaaagatgaccaacaaggtggttgaatccgccaaggccatcctggggggctcaaaggtgcgggtcgatcagaaatgtaaatcctctgttcaaattgattctacccctgtcattgtaacttccaatacaaacatgtgtgtggtggtggatgggaattccacgacctttgaacaccagcagccgctggaggaccgcatgttcaaatttgaactgactaagcggctcccgccagattttggcaagattactaagcaggaagtcaaggacttttttgcttgggcaaaggtcaatcaggtgccggtgactcacgagtttaaagttcccagggaattggcgggaactaaaggggcggagaaatctctaaaacgcccactgggtgacgtcaccaatactagctataaaagtctggagaagcgggccaggctctcatttgttcccgagacgcctcgcagttcagacgtgactgttgatcccgctcctctgcgaccgctcaattggaattcaaggtatgattgcaaatgtgactatcatgctcaatttgacaacatttctaacaaatgtgatgaatgtgaatatttgaatcggggcaaaaatggatgtatctgtcacaatgtaactcactgtcaaatttgtcatgggattcccccctgggaaaaggaaaacttgtcagattttggggattttgacgatgccaataaagaacagtaaataaagcgagtagtcatgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctcttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaacccattcatgtcgcataccctcaataaaccgtgtattcgtgtcagtaaaatactgcctcttgtggtcattcaatgaataacagcttacaacatctacaaaacctccttgcttgagagtgtggcactctcccccctgtcgcgcg

(19) United States
(12) Patent Application Publication
(10) Pub. No.: US 2005/0106125 A1
(43) Pub. Date: May 19, 2005

(54) Use of AAV integration efficiency element for mediating site-specific integration of a transcription unit

(75) Inventors: Erik S. Falck-Pedersen, Dobbs Ferry, NY (US); Nicola Philpott, London (GB)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Cornell Research Foundation, Inc., Ithaca, NY (US)

(21) Appl. No.: 10/959,017

(22) Filed: Oct. 5, 2004

Related U.S. Application Data
(63) Continuation of application No. PCT/US03/11191, filed on Apr. 9, 2003.
(60) Provisional application No. 60/371,044, filed on Apr. 9, 2002.

(51) Int. Cl. A61K 48/00; C12N 15/861

(52) U.S. Cl. 424/93.2; 435/456; 435/325

(57) ABSTRACT

The invention provides an expression construct comprising a nucleic acid sequence encoding an adeno-associated virus integration efficiency element (AAV IEE), wherein the expression construct is substantially devoid of AAV inverted terminal repeats (AAV ITRs). Such an expression construct site-specifically integrates into a host cell chromosome when provided to a host cell in conjunction with an AAV Rep protein. The invention also provides a method of integrating a nucleic acid sequence of interest into a host cell chromosome through use of such an expression construct, as well as a method of prophylactically or therapeutically treating a mammal for a pathologic state comprising administering to a mammal such an expression construct comprising a nucleic acid sequence encoding a therapeutic factor.

### CROSS-REFERENCE TO RELATED PATENT APPLICATIONS

This patent application is a continuation of copending International Patent Application No. PCT/US03/11191, filed Apr. 9, 2003, which designates the United States, and which claims the benefit of U.S. Provisional Patent Application No. 60/371,044, filed Apr. 9, 2002.

### STATEMENT AS TO RIGHTS TO INVENTIONS MADE UNDER FEDERALLY SPONSORED RESEARCH AND DEVELOPMENT

This invention was made in part with United States Government support under Grant Number HL59312 awarded by the National Heart, Lung, and Blood Institute of the National Institutes of Health. The United States Government may have certain rights in this invention.

### TECHNICAL FIELD OF THE INVENTION

The invention pertains to an expression construct containing a nucleic acid sequence encoding an adeno-associated integration efficiency element to achieve site-specific integration of a nucleic acid sequence of interest into a host cell genome.

### BACKGROUND OF THE INVENTION

Gene therapy is emerging as a popular form of treatment that aims to address a variety of disease states through the transfer of functional genetic material into cells. Critical to the success of gene therapy is the development of safe and efficient gene transfer vehicles. To date, various strategies have been developed for the transfer of therapeutic genes and include both viral and nonviral means. All of these gene delivery systems, however, suffer from limitations in their applicability and efficacy (see, e.g., Verma et al., Nature, 389:239-242 (1997)). The most commonly used gene transfer vehicles have been of viral origin (e.g., adenovirus, retrovirus, adeno-associated virus).

Adenovirus (Ad) vectors provide efficient means of transgene delivery to a variety of cell types, regardless of mitotic state. They do, however, produce only transient expression (no integration and no replication if replication-deficient) in vivo and may cause adverse reactions when administered. Retroviruses offer the desirable feature of being able to insert a gene of interest into the host genome, thus contributing to the stability of the transduced gene. However, retroviruses have a limited host range, and successful infection occurs only in mitotic cells, with the exception of the human immunodeficiency virus. Additionally, retroviruses integrate randomly into the host cell chromosome, thus raising some concern about the potential activation of transcriptionally silent oncogenes and the possible inactivation of tumor suppressor genes mediated by insertional mutagenesis. Adeno-associated viral (AAV) vectors, however, have not been associated with any human disease and are capable of site-specific integration into the host genome. The major disadvantages associated with AAV vectors are their inability to carry large transgene sequences and the relatively low yield of recombinant vector produced by current methods. In view of these limitations, there have been many attempts to combine the site-specific integration properties of AAV vectors with other vector systems, such as Ad vectors (see, e.g., U.S. Pat. No.5,856,152) and baculovirus (see, e.g., Palombo et al., J. Virol., 72(6):5025-5034 (1998), as well as with nonviral delivery systems, such as liposomes (see, e.g., Lamartina et al., J. Virol., 72(9):7653-7658 (1998)).

Adeno-associated virus is a human parvovirus with a single-stranded DNA genome of 4.7 kb (see, e.g., Berns et al., Bioessays, 17:237-245 (1995)). It contains two open reading frames (ORFs), Rep and Cap, which are flanked by two inverted terminal repeats (ITRs) (see, e.g., Kotin, Hum. Gene Ther., 5:793-801 (1994) and Srivastava, Intervirology, 27:138-147 (1987)). The ITRs are 160 nucleotides in length and are considered to be the only cis elements required for replication and site-specific integration of the AAV genome. In addition to the ITR elements, the Rep gene is necessary in trans to target the integration event to the AAVS1 site located on human chromosome 19 (see, e.g., Balague et al., J. Virol., 71:3299-3306 (1997), Bertran et al., Ann. NY Acad. Sci., 850:163-177 (1998), Lamartina et al. (1998), supra, Pieroni et al., Virology, 249:249-259 (1998), Shelling et al., Gene Ther., 1:165-169 (1994), and Surosky et al., J. Virol., 71:7951-7959 (1997)).

The Rep ORF encodes four non-structural proteins, Rep 40, 52, 68, and 78, which are involved in the replication of the AAV genome (see, e.g., Srivastava et al., J. Virol., 45:555-564 (1983), and Trempe et al., Virology, 161:18-28 (1987)). Rep 68 and 78 are gene products of alternatively spliced mRNA transcribed from the AAV p5 promoter. These larger Rep proteins have DNA binding, site-specific and strand-specific endonuclease activities, as well as ATPase and DNA-DNA and DNA-RNA helicase functions (see, e.g., Im et al., Cell, 61:447-457 (1990), Im et al., J. Virol., 66:1119-1128 (1992), Wonderling et al., J. Virol., 70:4783-4786 (1996), and Zhou et al., J. Virol., 73:1580-1590 (1999)). Transcription from the p19 promoter generates Rep 52 and Rep 40. Rep 52 is known to have helicase and ATPase activities but no DNA binding or endonuclease activity (see, e.g., Smith et al., J. Virol., 72:4874-4881 (1998)).

As mentioned previously, AAV is the only known virus that site-specifically integrates into the human genome. Such site-specific integration occurs at the AAVS1 site at position 19q13.3 located on human chromosome 19 (see, e.g., Giraud et al., PNAS, 91:10039-10043 (1994), Kotin et al., PNAS, 87:2211-2215 (1990), and Samulski et al., EMBO J., 10:3941-3950 (1991)). Although the precise molecular mechanisms of AAV site-specific integration are not well understood, it appears that Rep 68 and 78 are critical. Indeed, it is thought that these proteins function by binding to the Rep Binding Elements (RBEs) situated in both AAV ITRs and at the AAVS1 site (see, e.g., Chiorini et al., Hum. Gene Ther., 6:1531-1541 (1995), Giraud et al. (1994), supra, and Weitzman et al., J. Virol., PNAS, 91:5808-5812 (1994)). The endonuclease activity of the two larger Rep proteins allows them to nick at the terminal resolution site (trs), which is positioned 8 bp or 11 bp away from the RBEs in AAV and AAVS1, respectively. An interaction between Rep molecules that are bound to the AAV genome with those that are bound to the AAVS1 site then takes place, and a nonhomologous recombination event occurs, resulting in integration of the AAV genome (see, e.g., Linden et al., PNAS, 93:11288-11294 (1996), Linden et al., PNAS, 93:7966-7972 (1996), Urcelay et al., J. Virol., 69:2038-2046 (1995), and Yang et al., J. Virol., 71:9231-9247 (1997)). It has been shown that head-to-tail concatemers of the wild-type AAV genome are able to site-specifically integrate in this manner (see, e.g., Giraud et al., J. Virol., 69:6917-6924 (1995)).

There have been numerous attempts to modify current vector systems to include the site-specific integration properties of AAV. A majority of these strategies have involved incorporating an expression cassette comprising a transgene flanked by at least one AAV ITR into a replication deficient Ad vector. When administered to a mammal in conjunction with an AAV Rep protein provided in trans, the expression cassette integrates into the mammalian genome. Other attempts have involved creating recombinant AAV (rAAV) vectors by modifying (e.g., mutating) one or more of the AAV ITRs. For example, International Patent Application WO 99/64569 describes methods and compositions for generating rAAV vectors. The methods generally comprise providing a helper plasmid encoding Rep and Cap polypeptides, providing a rAAV plasmid, which generally comprises a heterologous nucleotide sequence flanked by two AAV ITRs, and introducing both into a host cell. The distinction of this method, as compared to the earlier methods, purportedly is that there is no distal D-sequence homology between the helper plasmid and the rAAV plasmid. The D-sequence is contained in the AAV ITRs and is comprised of a stretch of 20 nucleotides. The '569 PCT application contends that the distal 10 nucleotides of the D-sequence are responsible for wild-type AAV contamination and that the proximal 10 nucleotides are necessary and sufficient to mediate high-efficiency rescue, replication, and encapsidation of the viral genome in vivo. In that respect, the rAAV plasmid may be designed to lack the distal 10 nucleotides of the D-sequence.

Other experiments involving AAV vectors with mutant ITRs were performed by Young et al., PNAS, 98(24):13525-13530 (2001). These mutants contain a nonfunctional terminal resolution site (trs), which renders the AAV vectors incapable of supporting viral replication. The results presented by Young et al. demonstrate that, in the presence of Rep 78, both wild-type AAV and the trs mutant AAV vectors target to chromosome 19 with similar frequency. Young et al. thus suggest that a functional ITR, as defined by AAV lytic replication, is not required for targeted integration. In that respect, these AAV vectors can be designed to contain mutant ITRs that are defective for AAV Rep-dependent replication but are completely competent for site-specific integration.

While previously described vectors have been somewhat effective at achieving site-specific integration, a need remains to provide an expression construct which is more efficient at integrating into a specific location into a host cell chromosome, such that these expression constructs can be formulated into therapeutic compositions and used in a method of treating a variety of pathologic states. The invention provides such a construct, composition, and method. These and other advantages of the invention, as well as additional inventive features, will be apparent from the description of the invention provided herein.

### SUMMARY OF THE INVENTION

The invention provides an expression construct comprising a nucleic acid sequence encoding an adeno-associated virus integration efficiency element (AAV IEE), wherein the expression construct is substantially devoid of AAV inverted terminal repeats (AAV ITRs). Such an expression construct site-specifically integrates into a host cell chromosome when provided to a host cell in conjunction with an AAV Rep protein. The invention also provides a method of integrating a nucleic acid sequence of interest into a host cell chromosome. The method comprises providing to a host cell an expression construct comprising a nucleic acid sequence encoding an AAV IEE and a nucleic acid sequence of interest, wherein the expression construct is substantially devoid of AAV ITRs, and providing to the host cell a nucleic acid sequence encoding an AAV Rep protein. The nucleic acid sequence encoding the AAV Rep protein is expressed, thereby resulting in the site-specific integration of the nucleic acid sequence of interest into a chromosome contained in the host cell. Further provided by the invention is a method of prophylactically or therapeutically treating a mammal for a pathologic state. The method comprises administering to a mammal an expression construct comprising a nucleic acid sequence encoding an AAV IEE and, a nucleic acid sequence encoding a therapeutic factor, wherein the expression construct is substantially devoid of AAV ITRs, and administering to the mammal a nucleic acid sequence encoding an AAV Rep protein. The nucleic acid sequence encoding the AAV Rep protein is expressed, thereby resulting in the site-specific integration of the nucleic acid sequence encoding a therapeutic factor into a chromosome of a cell of the mammal, the expression of the nucleic acid sequence encoding the therapeutic factor, and the subsequent production of the therapeutic factor to prophylactically or therapeutically treat the mammal for the pathologic state.

### BRIEF DESCRIPTION OF THE DRAWINGS

FIG. 1 sets forth the nucleotide sequence encoding an adeno-associated integration efficiency element isolated from the adeno-associated virus of serotype 2 (SEQ ID NO:1).

FIG. 2 sets forth the homology of AAV IEE isolated from the adeno-associated virus of serotype 2 with regions of the adeno-associated virus of serotypes 1 (SEQ ID NO:3), 3 (SEQ ID NO:4), 4 (SEQ ID NO:5), 5 (SEQ ID NO:6), and 6 (SEQ ID NO:2)).

### DETAILED DESCRIPTION OF THE INVENTION

The invention is predicated on the discovery of a nucleic acid sequence element which, when provided in collaboration with the necessary Rep proteins, is sufficient for Rep-mediated site-specific integration into a host cell chromosome. The invention is contrary to the currently held belief that site-specific integration is possible only in the presence of at least one adeno-associated virus inverted terminal repeat (AAV ITR). Accordingly, the invention provides an expression construct comprising a nucleic acid sequence encoding an adeno-associated virus integration efficiency element (AAV IEE), wherein the expression construct is substantially devoid of AAV ITRs, and wherein the expression construct site-specifically integrates into a host cell chromosome when provided to the host cell in conjunction with an AAV Rep protein.

By “substantially devoid of AAV ITRs” is meant lacking at least the portion of an AAV ITR that is responsible for integration. AAV ITRs also are responsible for a number of other functions, including serving as the origins of replication for viral DNA synthesis. Thus, it is conceivable to include these regions in the expression construct but to exclude the regions(s) responsible for integration. Preferably, however, the AAV ITRs are completely removed from the expression constructs of the invention. The AAV IEE sequence can be included in an expression construct containing ITRs other than AAV ITRs (e.g., adenoviral ITRs).

AAV IEE is part of the p5 promoter region of AAV and is comprised of a nucleotide sequence containing various transcription factor-binding sites. In this respect, it is believed that AAV IEE uses cellular transcription factors (e.g., YY1) in collaboration with Rep 68 and/or Rep 78 to form an active integration complex. It is also believed that AAV IEE functions as a promoter for RNA polymerase II transcription of Rep 68 and 78 transcripts.

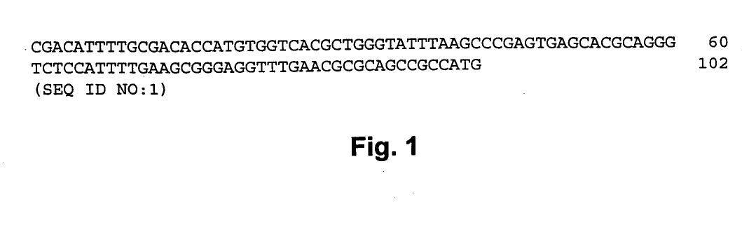

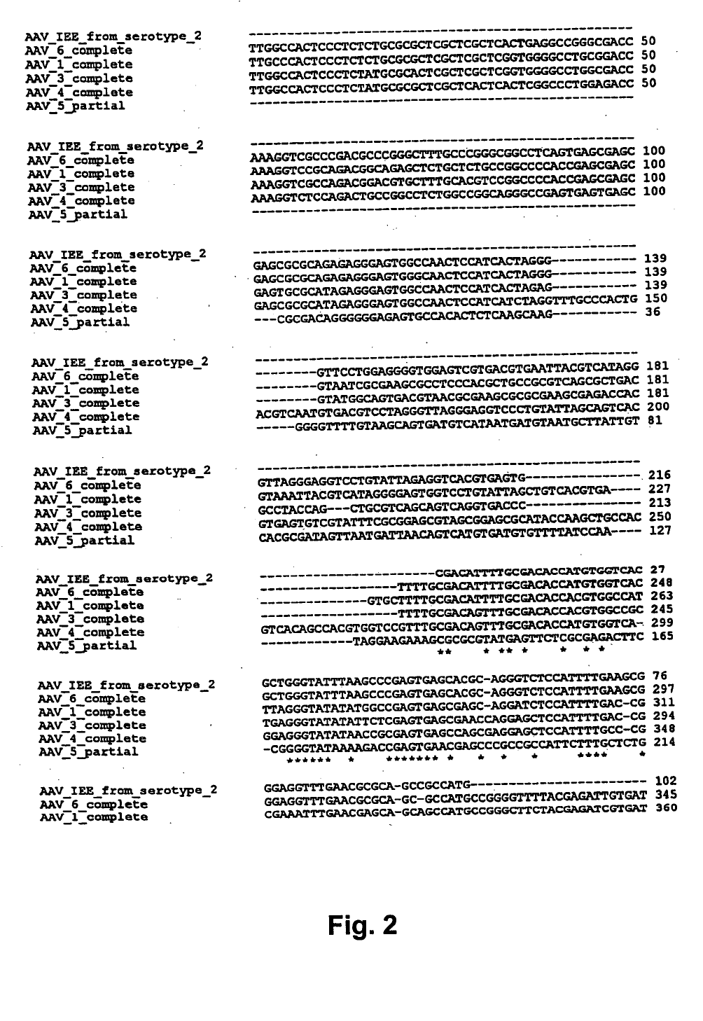

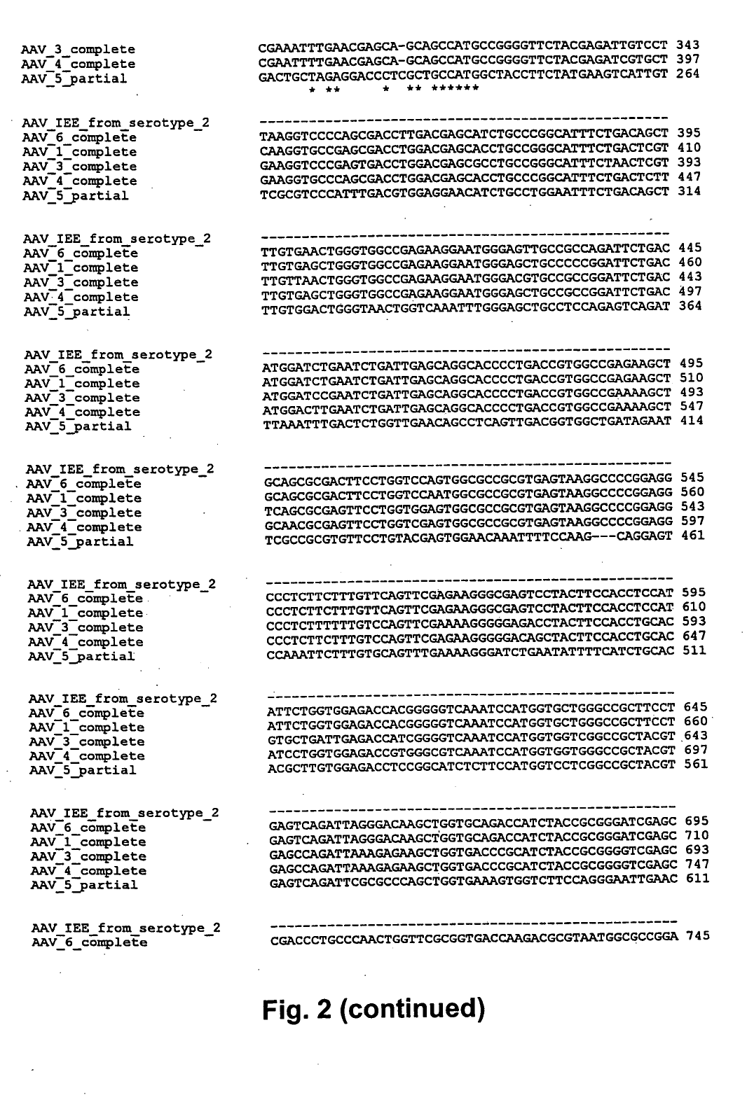

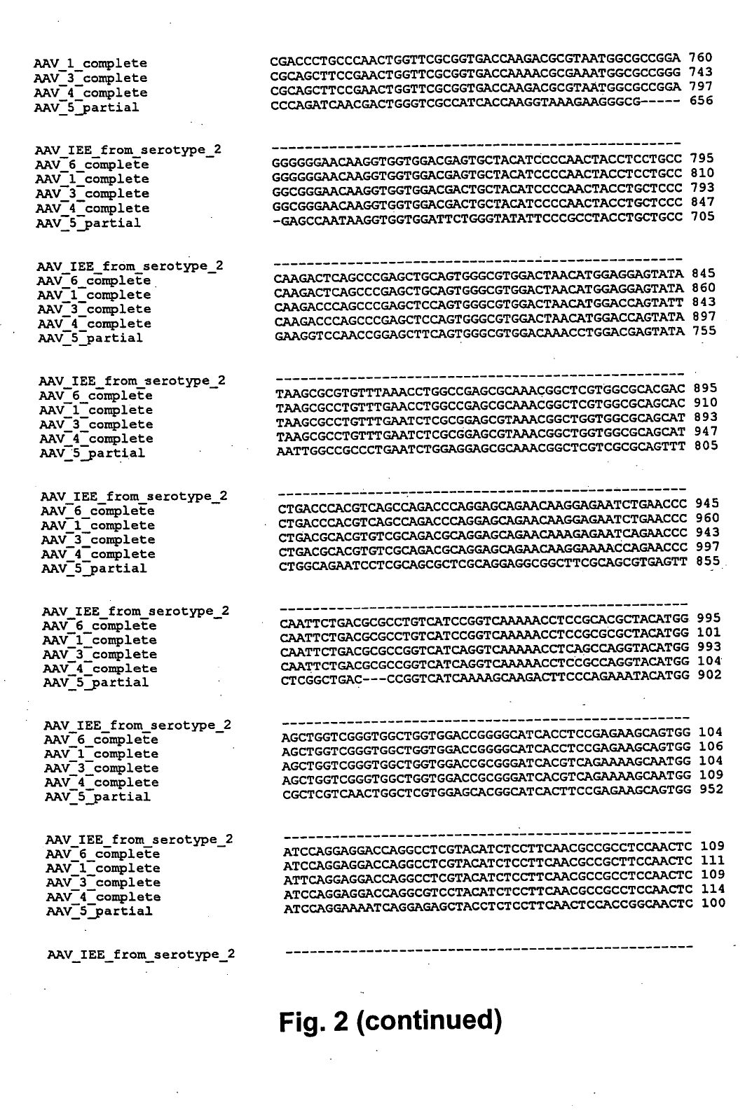

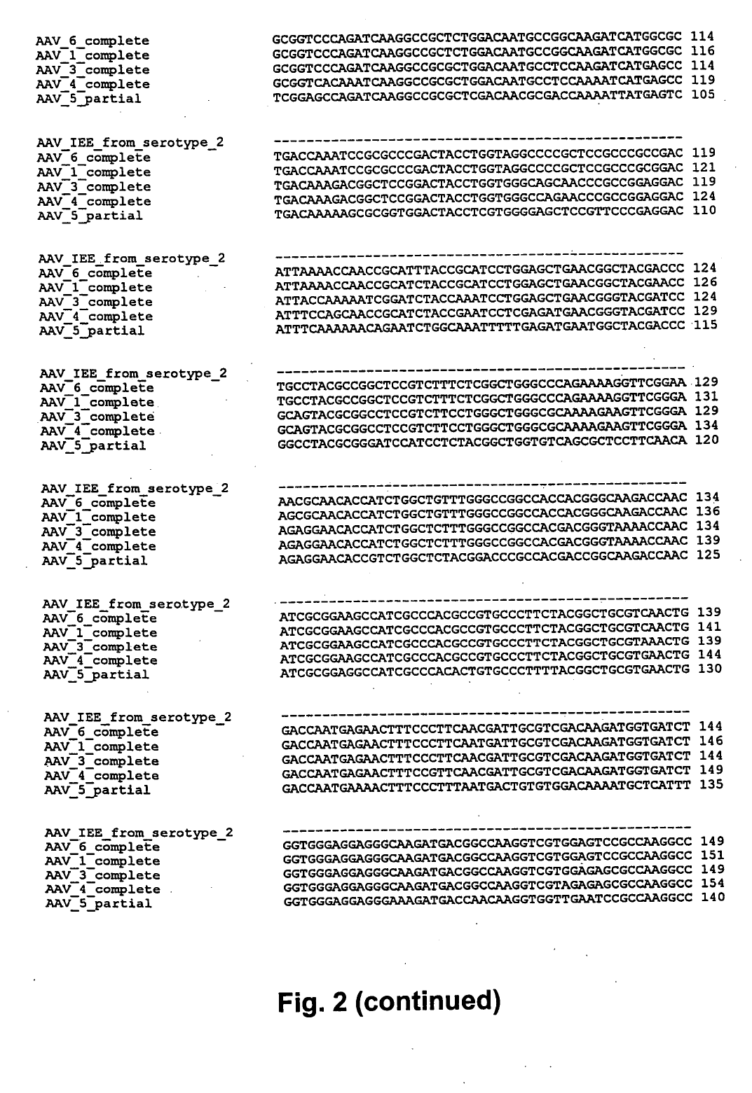

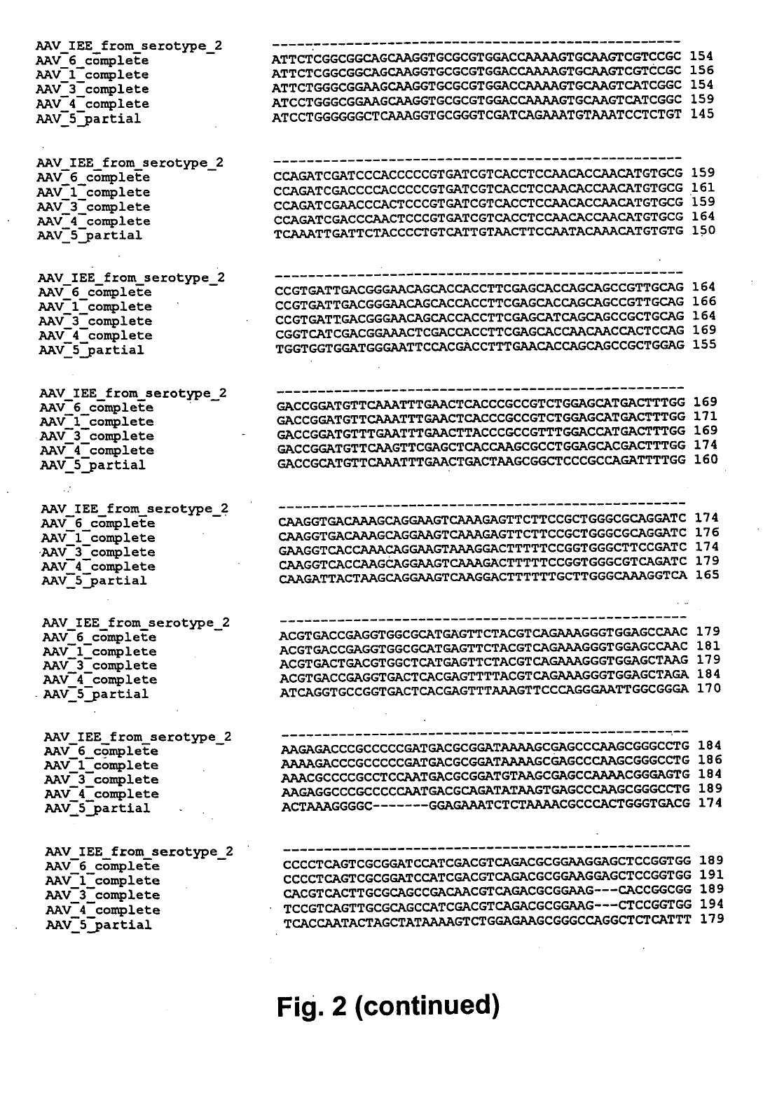

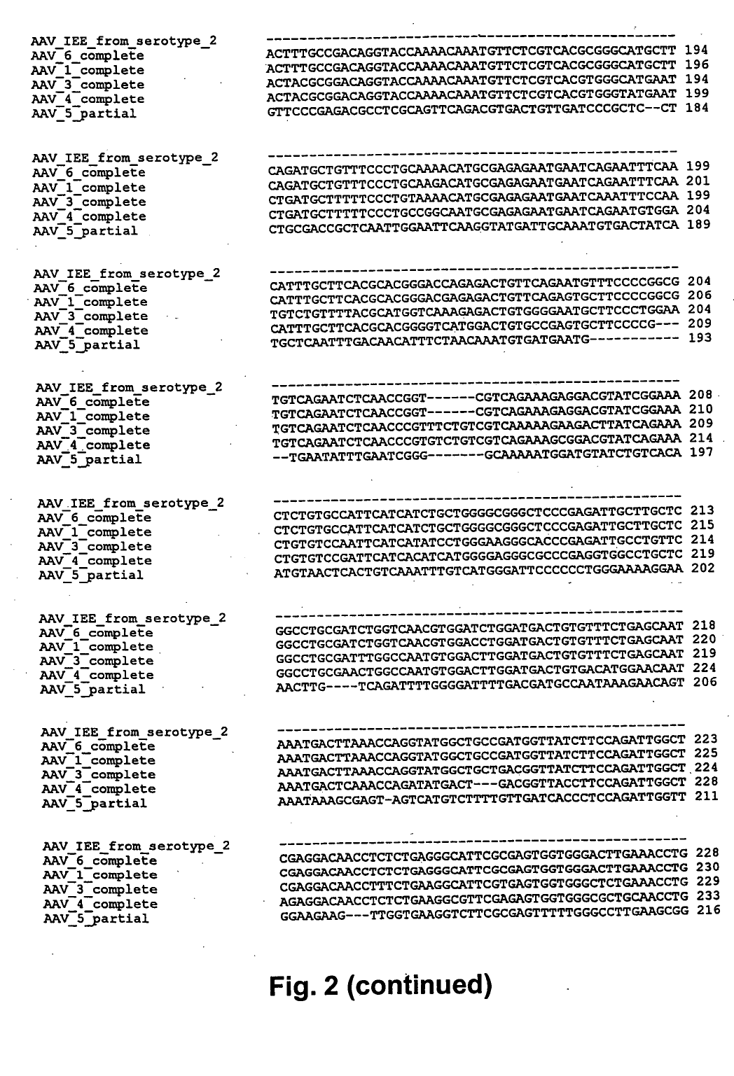

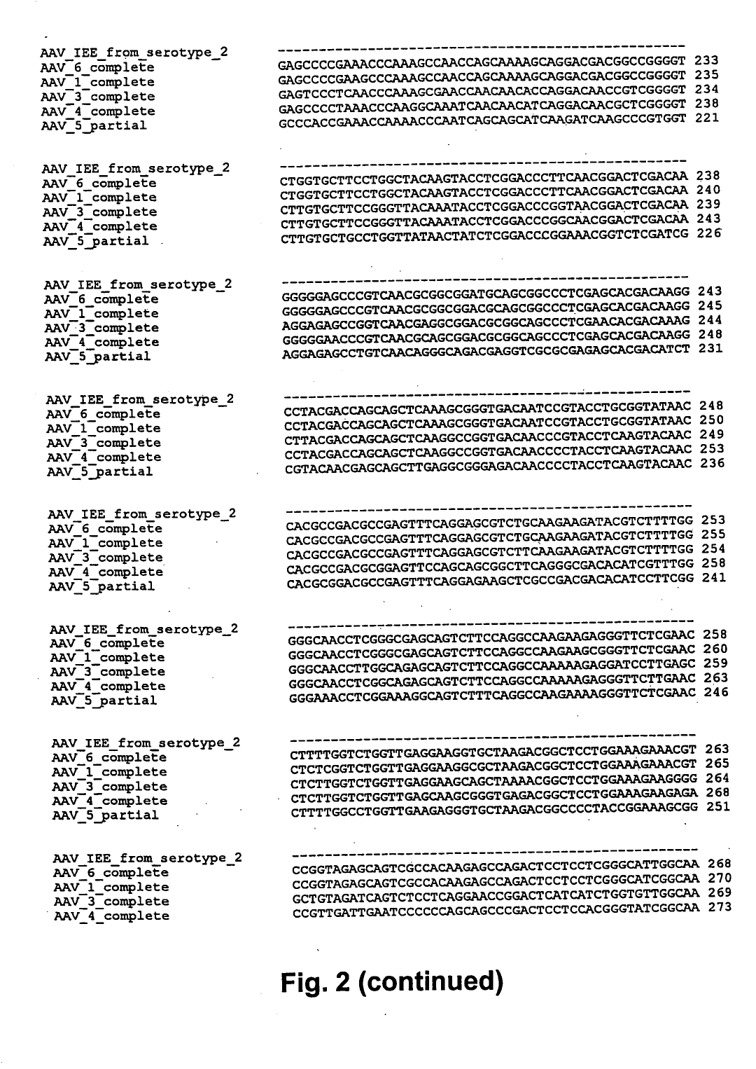

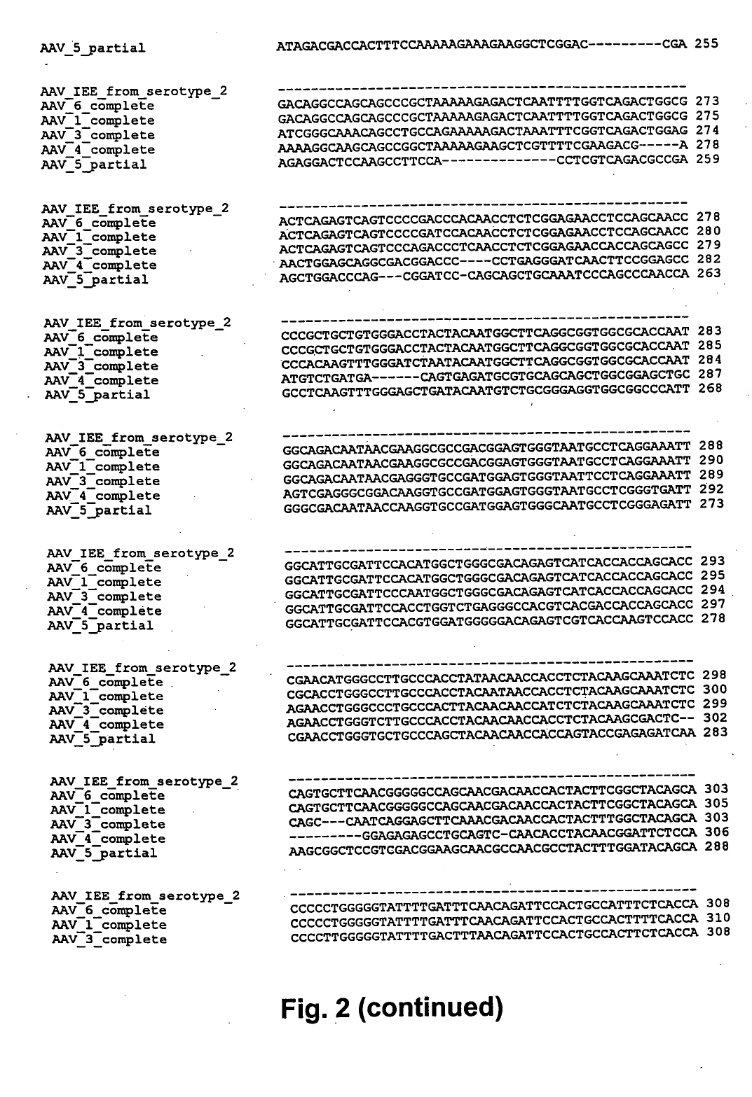

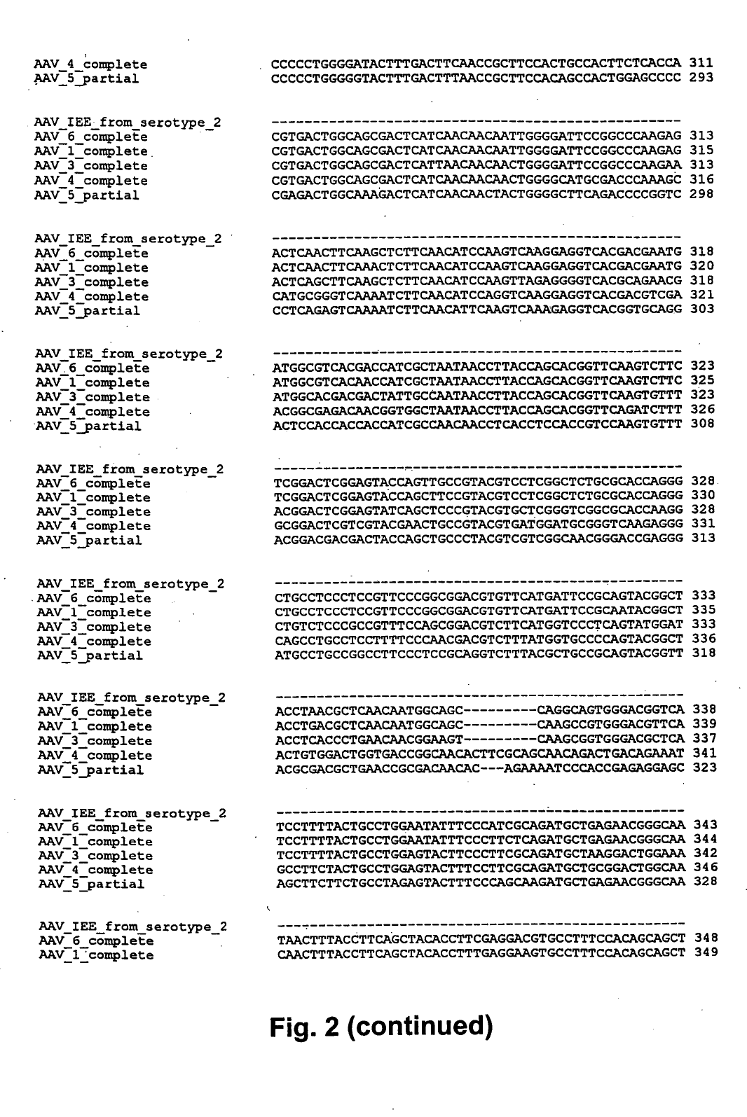

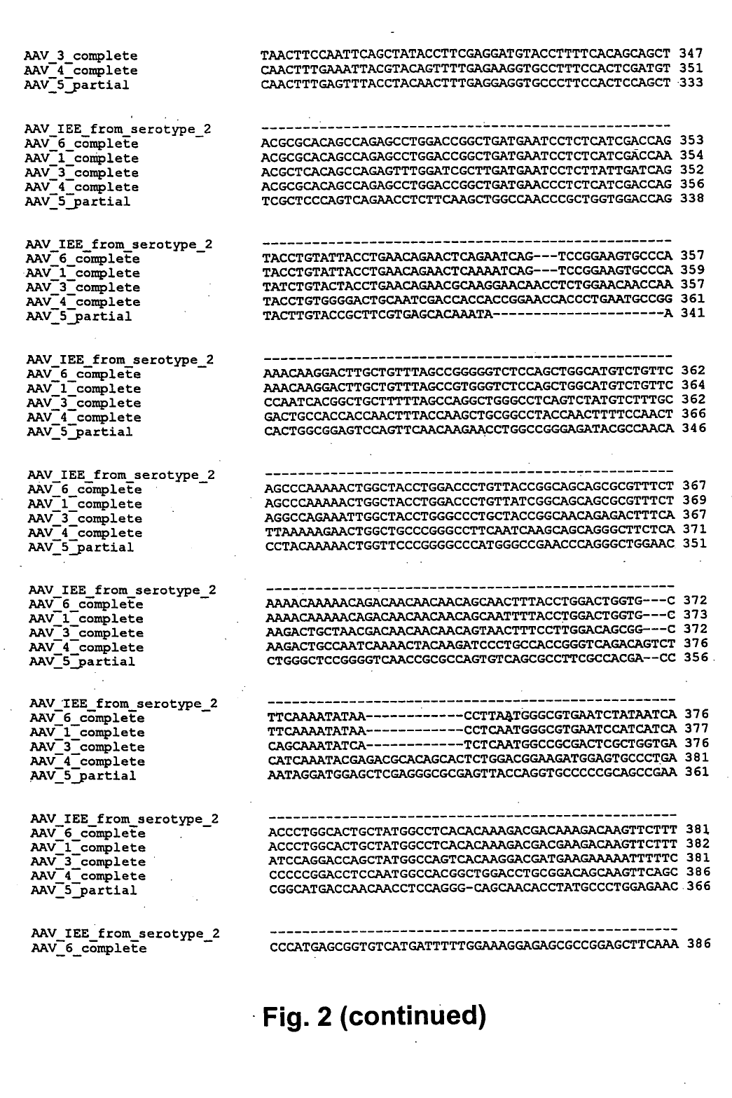

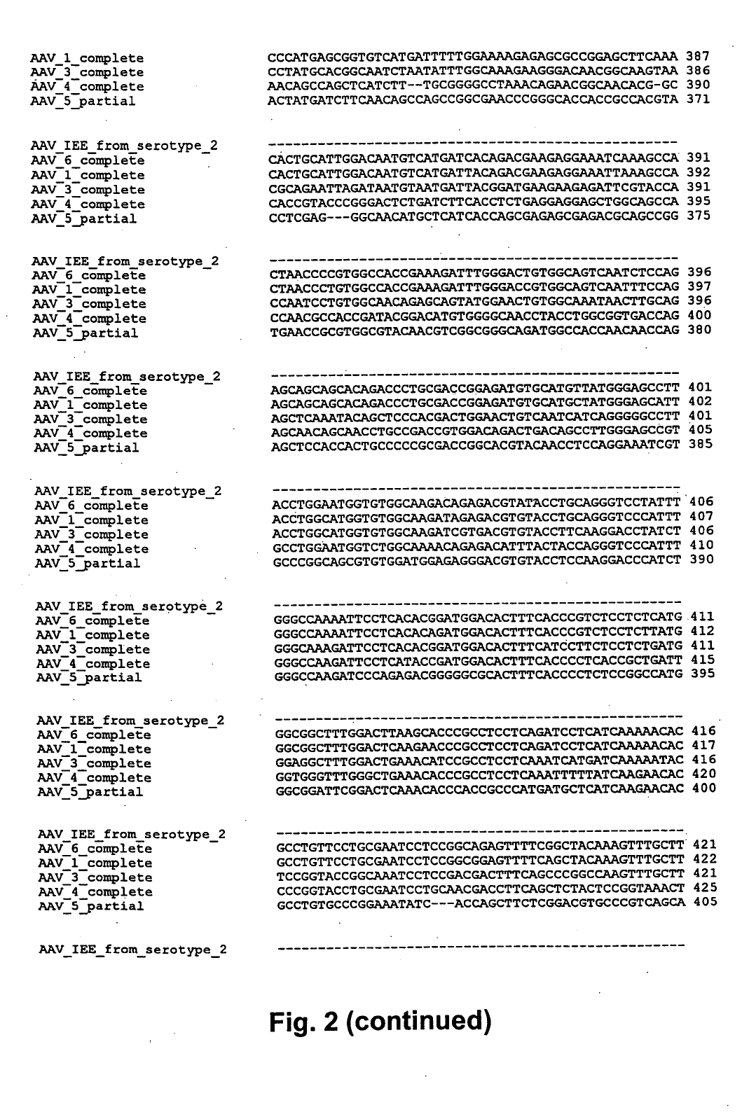

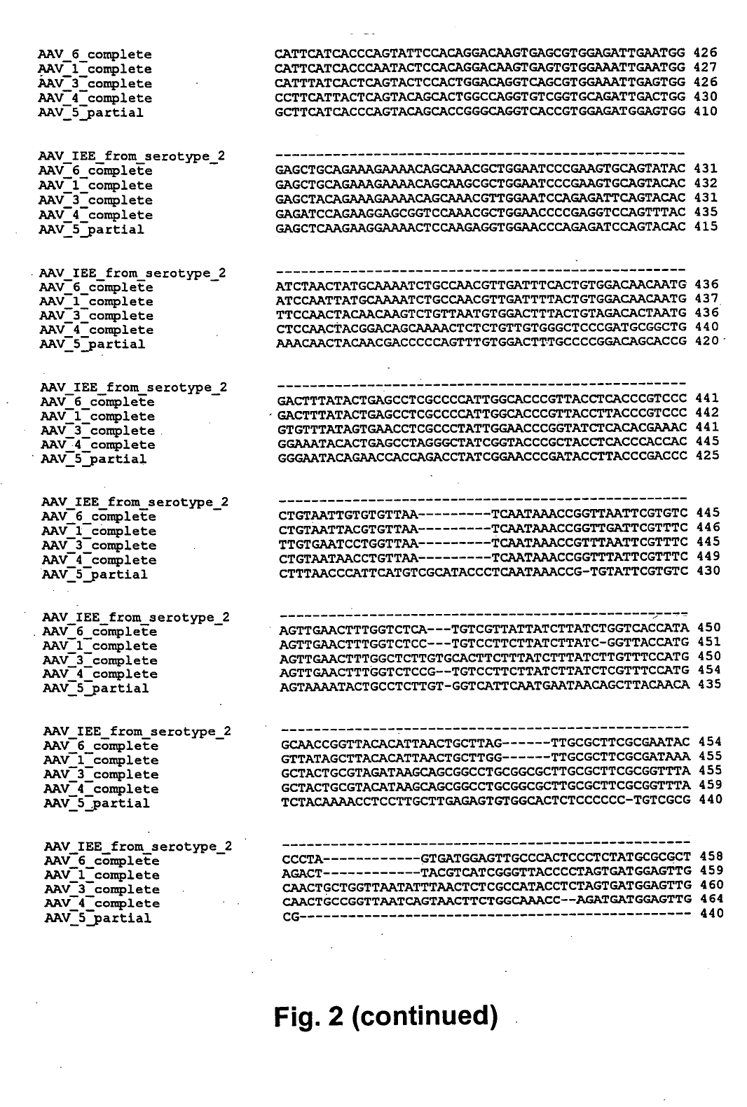

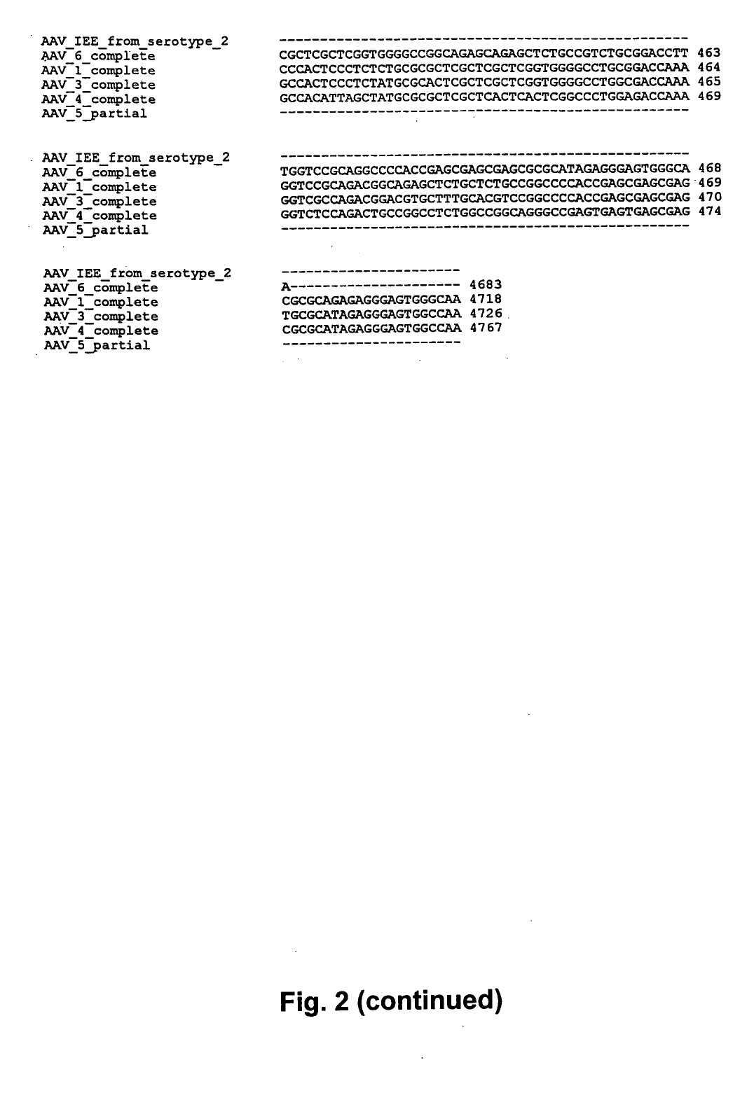

The nucleic acid sequence encoding an AAV IEE of the invention can be isolated from any AAV of the Dependovirus genus. For example, an AAV IEE can comprise nucleotides 222-324 in wild-type (wt) AAV of serotype 2 (AAV 2) (SEQ ID NO:1) (FIG. 1). This region, in particular, has been shown to be sufficient for directing site-specific integration in the absence of AAV ITRs. This nucleotide sequence also has shown to be homologous to regions contained in other AAV serotypes as well (e.g., serotypes 1 (SEQ ID NO:3), 3 (SEQ ID NO:4), 4 (SEQ ID NO:5), 5 (SEQ ID NO:6), and 6 (SEQ ID NO:2)) (FIG. 2), thus suggesting an AAV IEE exists in these other serotypes. Accordingly, a nucleic acid sequence encoding an AAV IEE, as it is referred to herein, is meant to encompass nucleotides 222-324 of wt AAV 2, homologous regions to this AAV 2 region in any other AAV serotype, such as AAV serotypes 1, 3, 4, 5, and 6, as well as fragments of any of the foregoing that direct site-specific integration. Performing routine experimentation can identify homologous regions and fragments suitable for use in the invention. Such experimentation will typically involve isolating nucleotides 222-324 of wt AAV 2, identifying homologous regions in other AAV serotypes, generating fragments of an AAV IEE from any AAV serotype, and assaying for activity of the homologous regions and fragments (e.g., assaying for the involvement of a particular homologous region or fragment in site-specific integration).

The term “host cell” denotes any cell that can be, or has been, used as a recipient of a nucleic acid sequence or an expression construct, and includes the progeny of the original cell which has been infected with the expression construct. These host cells must include a chromosome that allows site-specific integration of an expression construct of the invention. Thus, a host cell of the invention generally refers to a mammalian cell (e.g., a human cell). It is understood that the progeny of a single parental cell may not necessarily be completely identical in morphology or in genomic or total DNA complement as the original parent, due to natural, accidental, or deliberate mutation.

In the context of the invention, in terms of provision of the various aspects of the invention to cells, expression constructs are provided to a host cell, which is preferably a eukaryotic host cell. The eukaryotic host cell can be present in vitro or in vivo. According to the invention, “contacting” of cells with an expression construct of the invention can be by any suitable means by which the expression construct will be introduced into the cell. Preferably the expression constructs will be introduced by infection using the natural capability of the expression construct to enter cells (e.g., the capability of an expression construct to enter cells via receptor-mediated endocytosis). However, the expression constructs can be introduced by any other suitable means, e.g., transfection, calcium phosphate-mediated transformation, microinjection, electroporation, osmotic shock, and the like. Similarly, in a preferred embodiment of the invention, in vivo transfer of expression constructs is contemplated. Accordingly, the method of the invention also contemplates transfer in vivo by the methods set forth herein, or by any suitable method.

The AAV Rep protein used in the context of the invention can be any AAV Rep protein or combination of AAV Rep proteins, which is capable of providing the necessary function(s) to allow for site specific integration. Typically, AAV Rep proteins are characterized as being a short or a long form of Rep. The term “long forms of Rep” refers to the Rep 78 and/or Rep 68 gene products of the AAV Rep coding region, including functional homologues thereof. The long forms of Rep are normally expressed under the direction of the AAV p5 promoter. The term “short forms of Rep” refers to the Rep 52 and/or Rep 40 gene products of the AAV Rep coding region, including functional homologues thereof. The short forms of Rep are expressed under the direction of the AAV p19 promoter. Preferably, the AAV Rep protein comprises a long form of Rep (i.e., Rep 68 and/or Rep 78).

The AAV Rep protein can be provided to the host cell by any suitable means. For example, the AAV Rep protein can be provided to the host cell in trans. By “in trans” is meant being provided to the host cell via a different DNA molecule than the DNA molecule comprising the expression construct. However, it is also suitable for the AAV Rep protein to be provided to the host cell in the expression construct itself or, alternatively, incorporated into a packaging cell line. AAV Cap proteins also may be provided to the host cell by any suitable means, if desired. A number of vectors that contain the AAV Rep coding region are known, including those vectors described in U.S. Pat. No. 5,139,941, having ATCC accession numbers 53222, 53223, 53224, 53225 and 53226. Similarly, methods of obtaining vectors containing the HHV-6 homologue of AAV Rep are described in Thomson et al., Virology, 204:304-311 (1994). A number of vectors containing the AAV Cap coding region have also been described, including those vectors described in U.S. Pat. No. 5,139,941. Packaging cell lines derived from human 293 cells that have been transfected with a vector having the AAV Rep gene operably linked to a heterologous transcription promoter have been described in International Publication Nos. WO 95/13392 and WO 95/13365.

One of ordinary skill in the art will appreciate that any of a number of expression constructs known in the art are suitable for use in the invention. Examples of suitable expression constructs include, for instance, plasmids, plasmid-liposome complexes, and viral vectors. Any of these expression constructs are capable of being manipulated to include a nucleic acid sequence encoding an AAV IEE and can be prepared using standard recombinant DNA techniques described in, e.g., Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994). Alternatively, the nucleic acid sequence encoding an AAV IEE can be administered to a host cell as naked DNA.

Plasmids are genetically engineered circular double-stranded DNA molecules and can be designed to contain an expression cassette comprising an AAV IEE. Although plasmids were the first vector described for administration of therapeutic nucleic acids, the level of transfection efficiency is poor compared with other techniques. By complexing the plasmid with liposomes, the efficiency of gene transfer in general is improved. While the liposomes used for plasmid-mediated gene transfer strategies have various compositions, they are typically synthetic cationic lipids. Advantages of plasmid-liposome complexes include their ability to transfer large nucleic acid sequences and their relatively low immunogenicity. While plasmids are suitable for use in the invention, preferably the expression construct is a viral vector.

The viral vector can be any suitable viral vector. Suitable viral vectors include, but are not limited to, reoviruses, adenoviruses, adeno-associated based viruses, papovaviruses, parvoviruses, picornaviruses, and enteroviruses of any suitable origin (preferably of animal origin (e.g., avian or mammalian) and desirably of human origin). By “adeno-associated based viruses” is meant an expression construct containing any AAV derived gene(s) excluding the AAV ITRs. Other suitable viral vectors are known in the art and are well characterized. Examples of such viral vectors are described in, for example, Fields et al., VIROLOGY Lippincott-Raven (3rd ed. (1996) and 4th ed. (2000)); ENCYCLOPEDIA OF VIROLOGY, R. G. Webster et al., eds., Academic Press (2nd ed., 1999); FUNDAMENTAL VIROLOGY, Fields et al., eds., Lippincott-Raven (3rd ed., 1995); Levine, “Viruses,” Scientific American Library No. 37 (1992); MEDICAL VIROLOGY, D. O. White et al., eds., Academic Press (2nd ed. 1994); and INTRODUCTION TO MODERN VIROLOGY, Dimock, N.J. et al., eds., Blackwell Scientific Publications, Ltd. (1994). Preferably, the viral vector is derived from, or based on, a virus that normally infects animals, such as mammals (most preferably humans). Adenoviral (Ad) vectors based on human adenoviruses are preferred viral vectors.

Adenovirus is a 36 kb double-stranded DNA virus that efficiently transfers DNA in vivo to a variety of different target cell types. The Ad vector can be produced in high titers and can efficiently transfer DNA to replicating and non-replicating cells. The Ad vector genome can be generated using any species, strain, subtype, mixture of species, strains, or subtypes, or chimeric adenovirus as the source of vector DNA. Adenoviral stocks that can be employed as a source of adenovirus can be amplified from the adenoviral serotypes 1 through 51, which are currently available from the American Type Culture Collection (ATCC, Manassas, Va.), or from any other serotype of adenovirus available from any other source. For instance, an adenovirus can be of subgroup A (e.g., serotypes 12, 18, and 31), subgroup B (e.g., serotypes 3, 7, 11, 14, 16, 21, 34, and 35), subgroup C (e.g., serotypes 1, 2, 5, and 6), subgroup D (e.g., serotypes 8, 9, 10, 13, 15, 17, 19, 20, 22-30, 32, 33, 36-39, and 42-47), subgroup E (serotype 4), subgroup F (serotypes 40 and 41), or any other adenoviral serotype. Given that the human adenovirus serotype 5 (Ad5) genome has been completely sequenced, the adenoviral vector is described herein with respect to the Ad5 serotype. The Ad vector can be any adenoviral vector capable of growth in a cell, which is in some significant part (although not necessarily substantially) derived from or based upon the genome of an adenovirus. The Ad vector can be based on the genome of any suitable wild-type adenovirus. Preferably, the Ad vector is derived from the genome of a wild-type adenovirus of group C, especially of serotype 2 or 5. Ad vectors are well known in the art and are described in, for example, U.S. Pat. Nos. 5,559,099, 5,712,136, 5,731,190, 5,837,511, 5,846,782, 5,851,806, 5,962,311, 5,965,541, 5,981,225, 5,994,106, 6,020,191, and 6,113,913, International Patent Applications WO 95/34671, WO 97/21826, and WO 00/00628, and Thomas Shenk, “Adenoviridae and their Replication,” and M. S. Horwitz, “Adenoviruses,” Chapters 67 and 68, respectively, in Virology, B. N. Fields et al., eds., 3d ed., Raven Press, Ltd., New York (1996).

Preferably, the Ad vector is replication-deficient. By “replication-deficient” is meant that the Ad vector comprises a genome that lacks at least one replication-essential gene function. A deficiency in a gene, gene function, or gene or genomic region, as used herein, is defined as a deletion of sufficient genetic material of the viral genome to impair or obliterate the function of the gene whose nucleic acid sequence was deleted in whole or in part. Replication-essential gene functions are those gene functions that are required for replication (i.e., propagation) of a replication-deficient Ad vector. Replication-essential gene functions are encoded by, for example, the adenoviral early regions (e.g., the E1, E2, and E4 regions), late regions (e.g., the L1-L5 regions), genes involved in viral packaging (e.g., the IVa2 gene), and virus-associated RNAs (e.g., VA-RNA I and/or VA-RNA II). Preferably, the replication-deficient Ad vector comprises an adenoviral genome deficient in two or more gene functions required for viral replication. The two or more regions of the adenoviral genome are preferably selected from the group consisting of the E1, E2, and E4 regions. More preferably, the replication-deficient adenoviral vector comprises a deficiency in at least one replication-essential gene function of the E1 region (denoted an E1-deficient adenoviral vector). The E1 region of the adenoviral genome comprises the E1A region and the E1B region. The E1A and E1B regions comprise nucleic acid sequences coding for multiple peptides by virtue of RNA splicing. A deficiency of a gene function encoded by either or both of the E1A and/or E1B regions of the adenoviral genome (e.g., a peptide that performs a function required for replication) is considered a deficiency of a gene function of the E1 region in the context of the invention. In addition to such a deficiency in the E1 region, the recombinant adenovirus also can have a mutation in the major late promoter (MLP), as discussed in International Patent Application WO 00/00628. More preferably, the vector is deficient in at least one replication-essential gene function of the E1 region and at least part of the nonessential E3 region (e.g., an Xba I deletion of the E3 region) (denoted an E1/E3-deficient adenoviral vector).

Preferably, the adenoviral vector is “multiply deficient,” meaning that the adenoviral vector is deficient in one or more gene functions required for viral replication in each of two or more regions of the adenoviral genome. For example, the aforementioned E1-deficient or E1/E3-deficient Ad vector can be further deficient in at least one replication-essential gene function of the E4 region (denoted an E1/E4-deficient adenoviral vector). An adenoviral vector deleted of the entire E4 region can elicit a lower host immune response.

Alternatively, the Ad vector lacks replication-essential gene functions in all or part of the E1 region and all or part of the E2 region (denoted an E1/E2-deficient adenoviral vector). Ad vectors lacking replication-essential gene functions in all or part of the E1 region, all or part of the E2 region, and all or part of the E3 region also are contemplated herein. If the Ad vector is deficient in a replication-essential gene function of the E2A region, the vector preferably does not comprise a complete deletion of the E2A region, which is less than about 230 base pairs in length. Generally, the E2A region of the adenovirus codes for a DBP (DNA binding protein), a polypeptide required for DNA replication. DBP is composed of 473 to 529 amino acids depending on the viral serotype. It is believed that DBP is an asymmetric protein that exists as a prolate ellipsoid consisting of a globular Ct with an extended Nt domain. Studies indicate that the Ct domain is responsible for DBP's ability to bind to nucleic acids, bind to zinc, and function in DNA synthesis at the level of DNA chain elongation. However, the Nt domain is believed to function in late gene expression at both transcriptional and post-transcriptional levels, is responsible for efficient nuclear localization of the protein, and also may be involved in enhancement of its own expression. Deletions in the Nt domain between amino acids 2 to 38 have indicated that this region is important for DBP function (Brough et al., Virology, 196, 269-281 (1993)). While deletions in the E2A region coding for the Ct region of the DBP have no effect on viral replication, deletions in the E2A region which code for amino acids 2 to 38 of the Nt domain of the DBP impair viral replication. It is preferable that the multiply replication-deficient adenoviral vector contain this portion of the E2A region of the adenoviral genome. In particular, for example, the desired portion of the E2A region to be retained is that portion of the E2A region of the adenoviral genome which is defined by the 5′ end of the E2A region, specifically positions Ad5(23816) to Ad5(24032) of the E2A region of the adenoviral genome of serotype Ad5.

The Ad vector can be deficient in replication-essential gene functions of only the early regions of the adenoviral genome, only the late regions of the adenoviral genome, and both the early and late regions of the adenoviral genome. The adenoviral vector also can have essentially the entire adenoviral genome removed, in which case it is preferred that at least either the viral (i.e., adenoviral) inverted terminal repeats (Ad ITRs) and one or more promoters or the Ad ITRs and a packaging signal are left intact (i.e., an adenoviral amplicon). The larger the region of the adenoviral genome that is removed, the larger the piece of exogenous nucleic acid sequence that can be inserted into the genome. For example, given that the adenoviral genome is 36 kb, by leaving the Ad ITRs and one or more promoters intact, the exogenous insert capacity of the adenovirus is approximately 35 kb. Alternatively, a multiply deficient Ad vector that contains only an Ad ITR and a packaging signal effectively allows insertion of an exogenous nucleic acid sequence of approximately 37-38 kb. Of course, the inclusion of a spacer element in any or all of the deficient adenoviral regions will decrease the capacity of the adenoviral vector for large inserts. Suitable replication-deficient Ad vectors, including multiply deficient Ad vectors, are disclosed in U.S. Pat. Nos. 5,851,806 and 5,994,106 and International Patent Applications WO 95/34671 and WO 97/21826. An especially preferred adenoviral vector for use in the invention is that described in International Patent Application PCT/USO/20536.

It should be appreciated that the deletion of different regions of the Ad vector can alter the immune response of the mammal. In particular, the deletion of different regions can reduce the inflammatory response generated by the Ad vector. Furthermore, the Ad vector's coat protein can be modified so as to decrease the Ad vector's ability or inability to be recognized by a neutralizing antibody directed against the wild-type coat protein, as described in International Patent Application WO 98/40509.

The adenoviral vector, when multiply replication-deficient, especially in replication-essential gene functions of the E1 and E4 regions, preferably includes a spacer element to provide viral growth in a complementing cell line similar to that achieved by singly replication deficient Ad vectors, particularly an Ad vector comprising a deficiency in the E1 region. A spacer sequence is defined in the invention as any sequence of sufficient length to restore the size of the adenoviral genome to approximately the size of a wild-type adenoviral genome, such that the Ad vector is efficiently packaged into viral particles. The spacer element can contain any sequence or sequences which are of the desired length. The spacer element sequence can be coding or non-coding and native or non-native with respect to the adenoviral genome, but does not restore the replication-essential function to the deficient region. The spacer can be of any suitable size, desirably at least about 15 base pairs (e.g., between about 15 base pairs and about 12,000 base pairs), preferably about 100 base pairs to about 10,000 base pairs, more preferably about 500 base pairs to about 8,000 base pairs, even more preferably about 1,500 base pairs to about 6,000 base pairs, and most preferably about 2,000 to about 3,000 base pairs. The size of the spacer is limited only by the size of the insert that the Ad vector will accommodate (e.g., approximately 38 base pairs). In the absence of a spacer, production of fiber protein and/or viral growth of the multiply replication-deficient Ad vector is reduced by comparison to that of a singly replication-deficient Ad vector. However, inclusion of the spacer in at least one of the deficient adenoviral regions, preferably the E4 region, can counteract this decrease in fiber protein production and viral growth. The use of a spacer in an Ad vector is described in U.S. Pat. No. 5,851,806.

The Ad vector preferably contains a packaging domain. The packaging domain can be located at any position in the adenoviral genome, so long as the adenoviral genome is packaged into adenoviral particles. Preferably, the packaging domain is located downstream of the E1 region. More preferably, the packaging domain is located downstream of the E4 region. In a particularly preferred embodiment, the replication-deficient Ad vector lacks all or part of the E1 region and the E4 region. In this preferred embodiment, a spacer is inserted into the E1 region, a desired exogenous nucleic acid sequence of interest (e.g., a nucleic acid sequence encoding TNF-α) is located in the E4 region, and the packaging domain is located downstream of the E4 region. Thus, by relocating the packaging domain, the amount of potential overlap between the Ad vector and the cellular/helper virus genome is reduced.

The coat proteins of the Ad vector can be manipulated to alter the binding specificity of the resulting adenoviral particle. Suitable modifications to the coat proteins include, but are not limited to, insertions, deletions, or replacements in the adenoviral fiber, penton, pIX, pIIIa, pVI, or hexon proteins, or any suitable combination thereof, including insertions of various native or non-native ligands into portions of such coat proteins. Examples of Ad vectors with modified binding specificity are described in, e.g., U.S. Pat. Nos. 5,871,727, 5,885,808, and 5,922,315. Preferred modified Ad vector particles include those described in, for example, Wickham et al., J. Virol., 71(10), 7663-9 (1997), Cripe et al., Cancer Res., 61(7), 2953-60 (2001), van Deutekom et al., J. Gene Med., 1(6), 393-9 (1999), McDonald et al., J. Gene Med., 1(2), 103-10 (1999), Staba et al., Cancer Gene Ther., 7(1), 13-9 (2000), Wickham, Gene Ther., 7(2), 110-4 (2000), Kibbe et al., Arch. Surg., 135(2), 191-7 (2000), Harari et al., Gene Ther., 6(5), 801-7 (2000), Bouri et al., Hum Gene Ther., 10(10), 1633-40 (1999), Wickham et al., Nat. Biotechnol., 14(11), 1570-3 (1996), Wickham et al., Cancer Immunol. Immunother., 45(3-4), 149-51 (1997), and Wickham et al., Gene Ther., 2(10), 750-6 (1995), and U.S. Pat. Nos. 5,559,099; 5,712,136; 5,731,190; 5,770,442; 5,801,030; 5,846,782; 5,962,311; 5,965,541; 6,057,155; 6,127,525; and 6,153,435; and International Patent Applications WO 96/07734, WO 96/26281, WO 97/20051, WO 98/07865, WO 98/07877, WO 98/40509, WO 98/54346, WO 00/15823, and WO 01/58940.

Replication-deficient Ad vectors are typically produced in complementing cell lines that provide gene functions not present in the replication-deficient Ad vectors, but required for viral propagation, at appropriate levels in order to generate high titers of viral vector stock. A preferred cell line complements for at least one and preferably all replication-essential gene functions not present in a replication-deficient adenovirus. The complementing cell line can complement for a deficiency in at least one replication-essential gene function encoded by the early regions, late regions, viral packaging regions, virus-associated RNA regions, or combinations thereof, including all adenoviral functions (e.g., to enable propagation of adenoviral amplicons, which comprise minimal adenoviral sequences, such as only Ad ITRs and the packaging signal or only Ad ITRs and an adenoviral promoter). Most preferably, the complementing cell line complements for a deficiency in at least one replication-essential gene function (e.g., two or more replication-essential gene functions) of the E1 region of the adenoviral genome, particularly a deficiency in a replication-essential gene function of each of the E1A and E1B regions. In addition, the complementing cell line can complement for a deficiency in at least one replication-essential gene function of the E2 (particularly as concerns the adenoviral DNA polymerase and terminal protein) and/or E4 regions of the adenoviral genome. Desirably, a cell that complements for a deficiency in the E4 region comprises the E4-ORF6 gene sequence and produces the E4-ORF6 protein. Such a cell desirably comprises at least ORF6 and no other ORF of the E4 region of the adenoviral genome. The cell line preferably is further characterized in that it contains the complementing genes in a non-overlapping fashion with the adenoviral vector, which minimizes, and practically eliminates, the possibility of the vector genome recombining with the cellular DNA. Accordingly, the presence of replication competent adenoviruses (RCA) is minimized if not avoided in the vector stock, which, therefore, is suitable for certain therapeutic purposes, especially gene therapy purposes. The lack of RCA in the vector stock avoids the replication of the Ad vector in non-complementing cells. The construction of complementing cell lines involves standard molecular biology and cell culture techniques, such as those described by Sambrook et al. (1989), supra, and Ausubel et al. (1984), supra. Complementing cell lines for producing the adenoviral vector include, but are not limited to, 293 cells (described in, e.g., Graham et al., J. Gen. Virol., 36, 59-72 (1977)), PER.C6 cells (described in, e.g., International Patent Application WO 97/00326, and U.S. Pat. Nos. 5,994,128 and 6,033,908), and 293-ORF6 cells (described in, e.g., International Patent Application WO 95/34671 and Brough et al., J. Virol., 71, 9206-9213 (1997)).

The selection of expression construct for use in the invention will depend on a variety of factors such as, for example, the host, immunogenicity of the expression construct, the desired duration of protein production, the target cell, and the like. As each type of expression construct has distinct properties, a researcher has the freedom to tailor the invention to any particular situation. Moreover, more than one type of expression construct can be used, if desired.

The expression construct of the invention can further comprise a nucleic acid sequence of interest. Preferably, the nucleic acid sequence of interest is an exogenous nucleic acid sequence and encodes a protein. The nucleic acid sequence of interest can encode any protein that is desired for site-specific integration into a host cell genome. Preferably, the protein is a therapeutic factor useful to prophylactically or therapeutically treat a mammal for a pathologic state. For example, the therapeutic factor can be a cytokine. Examples of cytokines include, but are not limited to, interleukins, interferons (i.e., INF-α, INF-β, INF-γ), leukemia inhibitory factor (LIF), oncostatin M (OSM), granulocyte-macrophage colony stimulating factor (GM-CSF), granulocyte colony stimulating factor (G-CSF), tumor necrosis factor-alpha (TNF-α), tumor necrosis factor-beta (TNF-β), and transforming growth factor-beta (TGF-β). Preferably, the cytokine is selected from the group consisting of an interleukin, an interferon and a tumor necrosis factor. The therapeutic factor also can be a protein that is toxic to a specific subset of cells. In this respect, the nucleic acid sequence can encode an apoptotic factor (e.g., Bax, Bak, Bcl-Xₛ, Bad, Bim, Bik, Bid, Harakiri, ICE-CED3 proteases, TRAIL, SARP-2, apoptin); an enzyme (e.g., cytosine deaminase, adenosine deaminase, hypoxanthine-guanine phosphoribosyltransferase, and thymidine kinase); a toxin (e.g., ricin A-chain, diphtheria toxin A, pertussis toxin A subunit, E. coli enterotoxin A subunit, cholera toxin A subunit and pseudomonas toxin c-terminal); an antisense molecule; a ribozyme; or a cell cycle regulator (e.g., p27, p21, p57, p18, p73, p19, p15, E2F-1, E2F-2, E2F-3, p107, p130 and E2F-4). Other therapeutic factors include those involved in the promotion or inhibition of angiogenesis. Alternatively, the nucleic acid sequence of interest can be useful for creating a stable host cell line wherein the nucleic acid sequence is permanently integrated into a specific location of the host cell genome.

In view of the above, the invention also provides a method of integrating a nucleic acid sequence of interest into a host cell chromosome. The method comprises (a) providing to a host cell an expression construct comprising a nucleic acid sequence encoding an AAV IEE and a nucleic acid sequence of interest, wherein the expression construct is substantially devoid of AAV ITRs, and (b) providing to the host cell a nucleic acid sequence encoding an AAV Rep protein, such that the nucleic acid sequence encoding the AAV Rep protein is expressed, thereby resulting in the site-specific integration of the nucleic acid sequence of interest into a chromosome contained in the host cell. Such a method can be useful for creating stable cell lines as well as for therapeutic purposes. For example, a host cell can be contacted with an expression construct as described above and then propagated to produce a stable host cell line. Such a cell line will have the nucleic acid sequence of interest permanently integrated into its genome in a specific location. Depending on the host cell, various propagation techniques can be employed. Host cell propagation is well within the skill in the art and is described in, for example, Sambrook et al. (1989), supra.

The nucleic acid sequence of interest can be located at any suitable position in the expression construct. For example, the nucleic acid sequence can be positioned upstream of an AAV IEE. Preferably, however, the nucleic acid sequence of interest is positioned downstream of an AAV IEE in the expression construct. For the purpose of describing the relative position of nucleotide sequences in an expression construct throughout the instant application, such as when a particular nucleic acid sequence is described as being situated “upstream,” “downstream,” “3′,” or “5′” relative to another sequence, it is to be understood that it is the position of the sequences in the “sense” or “coding” strand of a DNA molecule that is being referred to as is conventional in the art.

According to the invention, the nucleic acid sequence of interest is operably linked to regulatory sequences necessary for expression, i.e., a promoter. A “promoter” is a DNA sequence that directs the binding of RNA polymerase and thereby promotes RNA synthesis. A nucleic acid sequence is “operably linked” to a promoter when the promoter is capable of directing transcription of that nucleic acid sequence. A promoter can be native or non-native to the nucleic acid sequence to which it is operably linked.

Any promoter (i.e., whether isolated from nature or produced by recombinant DNA or synthetic techniques) can be used in connection with the invention to provide for transcription of a particular nucleic acid sequence. The promoter preferably is capable of directing transcription in a eukaryotic (desirably mammalian) cell. The functioning of the promoter can be altered by the presence of one or more enhancers and/or silencers present on the vector. “Enhancers” are cis-acting elements of DNA that stimulate or inhibit transcription of adjacent genes. An enhancer that inhibits transcription also is termed a “silencer.” Enhancers differ from DNA-binding sites for sequence-specific DNA binding proteins found only in the promoter (which also are termed “promoter elements”) in that enhancers can function in either orientation, and over distances of up to several kilobase pairs (kb), even from a position downstream of a transcribed region.

The invention preferentially employs a viral promoter. Suitable viral promoters are known in the art and include, for instance, cytomegalovirus (CMV) promoters, such as the CMV immediate-early promoter, promoters derived from human immunodeficiency virus (HIV), such as the HIV long terminal repeat promoter, Rous sarcoma virus (RSV) promoters, such as the RSV long terminal repeat, mouse mammary tumor virus (MMTV) promoters, HSV promoters, such as the Lap2 promoter or the herpes thymidine kinase promoter (Wagner et al., PNAS, 78, 144-145 (1981)), promoters derived from SV40 or Epstein Barr virus, an adeno-associated viral promoter, such as the p5 promoter, and the like. Preferably, the viral promoter is an adenoviral promoter, such as the Ad2 or Ad5 major late promoter and tripartite leader, a CMV promoter, or an RSV promoter.

Many of the above-described promoters are constitutive promoters. Instead of being a constitutive promoter, the promoter can be an inducible promoter, i.e., a promoter that is up- and/or down-regulated in response to appropriate signals. Examples of suitable inducible promoter systems include, but are not limited to, the IL-8 promoter, the metallothionine inducible promoter system, the bacterial lacZYA expression system, the tetracycline expression system, and the T7 polymerase system. Further, promoters that are selectively activated at different developmental stages (e.g., globin genes are differentially transcribed from globin-associated promoters in embryos and adults) can be employed. The promoter sequence that regulates expression of the nucleic acid sequence can contain at least one heterologous regulatory sequence responsive to regulation by an exogenous agent. The regulatory sequences are preferably responsive to exogenous agents such as, but not limited to, drugs, hormones, or other gene products. For example, the regulatory sequences, e.g., promoter, preferably are responsive to glucocorticoid receptor-hormone complexes, which, in turn, enhance the level of transcription of a therapeutic peptide or a therapeutic fragment thereof.

One of ordinary skill in the art will appreciate that each promoter drives transcription, and, therefore, protein expression, differently with respect to time and amount of protein produced. For example, the CMV promoter is characterized as having peak activity shortly after transduction, i.e., about 24 hours after transduction, then quickly tapering off. On the other hand, the RSV promoter's activity increases gradually, reaching peak activity several days after transduction, and maintains a high level of activity for several weeks. Indeed, sustained expression driven by an RSV promoter has been observed in all cell types studied, including, for instance, liver cells, lung cells, spleen cells, diaphragm cells, skeletal muscle cells, and cardiac muscle cells. Thus, a promoter can be selected for use in the invention by matching its particular pattern of activity with the desired pattern and level of expression of a nucleic acid sequence of interest. Alternatively, a hybrid promoter can be constructed which combines the desirable aspects of multiple promoters. For example, a CMV-RSV hybrid promoter combining the CMV promoter's initial rush of activity with the RSV promoter's high maintenance level of activity would be especially preferred for use in many embodiments of the invention. It is also possible to select a promoter with an expression profile that can be manipulated by an investigator.

A nucleic acid sequence encoding a marker protein, such as green fluorescent protein or luciferase also can be present in the expression construct. Such marker proteins are useful in construction of the expression construct as well as in determining expression construct migration if administered to an organism. Marker proteins also can be used to determine points of injection in order to efficiently space injections of an expression construct composition to provide a widespread area of treatment, if desired. Alternatively, a nucleic acid sequence encoding a selection factor, which also is useful in vector construction protocols, can be part of the expression construct.

Negative selection genes can be incorporated into any of the above-described expression constructs. A preferred embodiment is an HSV tk gene cassette (Zjilstra et al., Nature, 342: 435 (1989); Mansour et al., Nature, 336: 348 (1988); Johnson et al., Science, 245: 1234 (1989): Adair et al., PNAS, 86: 4574 (1989); Capecchi, Science, 244: 1288 (1989)) operably linked to the E2 promoter. The tk expression cassette (or other negative selection expression cassette) is inserted into an adenoviral genome, for example, as a replacement for a substantial deletion of the E3 gene. Other negative selection genes will be apparent to those of skill in the art.

With respect to promoters, nucleic acid sequences, selectable markers, and the like, located on an expression construct according to the invention, such elements can be present as part of a cassette, either independently or coupled. In the context of the invention, a “cassette” is a particular base sequence that possesses functions, which facilitate subcloning, and recovery of nucleic acid sequences (e.g., one or more restriction sites) or expression (e.g., polyadenylation or splice sites) of particular nucleic acid sequences.

Construction of a nucleic acid sequence operably linked to regulatory sequences necessary for expression is well within the skill of the art (see, for example, Sambrook et al. (1989), supra). With respect to the expression of nucleic acid sequences according to the invention, the ordinary skilled artisan is aware that different genetic signals and processing events control levels of nucleic acids and proteins/peptides in a cell, such as, for instance, transcription, mRNA translation, and post-transcriptional processing. Transcription of DNA into RNA requires a functional promoter, as described herein.

Protein expression is dependent on the level of RNA transcription that is regulated by DNA signals, and the levels of DNA template. Similarly, translation of mRNA requires, at the very least, an AUG initiation codon, which is usually located within 10 to 100 nucleotides of the 5′ end of the message. Sequences flanking the AUG initiator codon have been shown to influence its recognition by eukaryotic ribosomes, with conformity to a perfect Kozak consensus sequence resulting in optimal translation (see, e.g., Kozak, J. Mol. Biol., 196: 947-950 (1987)). Also, successful expression of an exogenous nucleic acid in a cell can require post-translational modification of a resultant protein. Thus, production of a protein can be affected by the efficiency with which DNA (or RNA) is transcribed into mRNA, the efficiency with which mRNA is translated into protein, and the ability of the cell to carry out post-translational modification. These are all factors of which the ordinary skilled artisan is aware and is capable of manipulating-using standard means to achieve the desired end result.

Along these lines, to optimize protein production, preferably the nucleic acid sequence encoding a protein further comprises a polyadenylation site following the coding region of the nucleic acid sequence. Also, preferably all the proper transcription signals (and translation signals, where appropriate) will be correctly arranged such that the nucleic acid sequence will be properly expressed in the cells into which it is introduced. If desired, the nucleic acid sequence also can incorporate splice sites (i.e., splice acceptor and splice donor sites) to facilitate mRNA production. Moreover, if the nucleic acid sequence encodes a protein or peptide, which is a processed or secreted protein or acts intracellularly, preferably the nucleic acid sequence further comprises the appropriate sequences for processing, secretion, intracellular localization, and the like.

It will be appreciated that the expression construct can comprise multiple nucleic acid sequences of interest. For example, the expression construct can comprise multiple copies of a nucleic acid sequence encoding a therapeutic factor, each copy operably linked to a different promoter or to identical promoters. Moreover, any nucleic acid sequence encoding a therapeutic factor described herein can be altered from its native form to increase its therapeutic effect. For example, a cytoplasmic form of a therapeutic nucleic acid can be converted to a secreted form by incorporating a signal peptide into the encoded gene product.

The invention also provides a method of prophylactically or therapeutically treating a mammal for a pathologic state. The method comprises (a) administering to a mammal an expression construct comprising a nucleic acid sequence encoding an AAV IEE and a nucleic acid sequence encoding a therapeutic factor, wherein the expression construct is substantially devoid of AAV ITRs, and (b) administering to the mammal a nucleic acid sequence encoding an AAV Rep protein, such that the nucleic acid sequence encoding the AAV Rep protein is expressed, thereby resulting in the site-specific integration of the nucleic acid sequence encoding a therapeutic factor into a chromosome of a cell of the mammal, the expression of the nucleic acid sequence encoding a therapeutic factor, and the subsequent production of the therapeutic factor to prophylactically or therapeutically treat the mammal for the pathologic state.

By “prophylactic” is meant the protection, in whole or in part, against a pathologic state. By “therapeutic” is meant the amelioration, in whole or in part, of the pathologic state, itself, and/or the protection, in whole or in part, against further progression of the disease. One of ordinary skill in the art will appreciate that any degree of protection from, or amelioration of, a pathologic state is beneficial to a patient.

When used for therapeutic purposes, the expression construct of the invention can be purified from a host cell using a variety of conventional purification methods, such as CsCl gradients or chromatography (e.g., ion-exchange chromatography). Such purification techniques are well known and frequently practiced in the art.

An expression construct of the invention desirably is formulated and administered to a mammal in a composition (i.e., an expression construct composition). Such compositions typically comprise an expression construct and a carrier. Preferably, the carrier is a pharmaceutically (e.g., physiologically) acceptable carrier and can be used within the context of the invention. Such carriers are well known in the art. The choice of carrier will be determined, in part, by the particular site to which the composition is to be administered and the particular method used to administer the adenoviral vector composition.

Suitable formulations include aqueous and non-aqueous solutions, isotonic sterile solutions, which can contain anti-oxidants, buffers, bacteriostats, and solutes that render the formulation isotonic with the blood or intraocular fluid of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets of the kind previously described. When administering an expression construct composition, preferably the pharmaceutically acceptable carrier is a buffered saline solution. More preferably, the expression construct composition for use in the invention is administered in an expression construct composition formulated to protect the expression construct from damage prior to administration. For example, the expression construct composition can be formulated to reduce loss of the expression construct on devices used to prepare, store, or administer the expression construct composition, such as glassware, syringes, or needles. The expression construct composition can be formulated to decrease the light sensitivity and/or temperature sensitivity of the expression construct itself. To this end, the expression construct composition preferably comprises a pharmaceutically acceptable liquid carrier, such as, for example, those described above, and a stabilizing agent selected from the group consisting of polysorbate 80, L-arginine, polyvinylpyrrolidone, trehalose, and combinations thereof (see, e.g., U.S. Pat. No. 6,225,289). Use of such an expression construct composition will extend the shelf life of the expression construct composition, facilitate administration, and increase the effectiveness of the expression construct. In this regard, an expression construct composition also can be formulated to enhance transduction efficiency.

In addition, the composition of the invention can comprise, or alternatively can be co-administered with, other therapeutic or biologically active agents. By “co-administration” is meant administration before, concurrently with, e.g., in combination with the expression construct in the same formulation or in separate formulations, or after administration of the expression construct as described above. For example, nucleic acid sequences, proteins, and/or other agents useful in the treatment of a particular pathologic state can be present of co-administered with the composition of the invention. Suitable biologically active agents can include, for example, factors that control inflammation, such as ibuprofen or steroids, which can be co-administered to reduce swelling and inflammation associated with administration of the expression construct. Immunosuppressive agents can be co-administered to reduce inappropriate immune responses related to a disorder or the practice of the inventive method. Anti-angiogenic factors, such as soluble growth factor receptors, growth factor antagonists, i.e., angiotensin, and the like also can be co-administered, as well as can be neurotrophic factors. Similarly, vitamins and minerals, anti-oxidants, and micronutrients can be co-administered. Antibiotics, i.e., microbicides and fungicides, can be co-administered to reduce the risk of infection associated with a particular pathologic state. When treating cancer, other anticancer compounds can be used in conjunction with the composition of the invention and can include, but are not limited to, all of the known anticancer compounds approved for marketing in the United States and those that will become approved in the future. See, for example, Table 1 and Table 2 of Boyd, Current Therapy in Oncology, Section 1. Introduction to Cancer Therapy (J. E. Niederhuber, ed.), Chapter 2, by B. C. Decker, Inc., Philadelphia, 1993, pp. 11-22. More particularly, such other anticancer compounds can include doxorubicin, bleomycin, vincristine, vinblastine, VP-16, VW-26, cisplatin, carboplatin, procarbazine, and taxol for solid tumors in general; alkylating agents, such as BCNU, CCNU, methyl-CCNU and DTIC, for brain or kidney cancers; and antimetabolites, such as 5-FU and methotrexate, for colon cancer.

The pathologic state can be any pathologic state. For example, the pathologic state can be a disorder caused by an increased or decreased level of a particular gene product(s). By “increased level” is meant a level above that which is considered normal. Similarly, by “decreased level” is meant a level below that which is considered normal. Many cancers result from an increased level of an oncogene, or, alternatively, a decreased level of a tumor suppressor gene. Accordingly, the pathologic state preferably is cancer, and the nucleic acid sequence preferably encodes a therapeutic factor, which is toxic to one or more different cancer cell types.

The pathologic state can be any type of cancer. Cancers can include lung cancer, colon cancer, renal cancer, anal cancer, bile duct cancer, bladder cancer, bone cancer, brain cancer, spinal chord cancer, breast cancer, cervical cancer, lymphoma, endometrial cancer, esophageal cancer, gallbladder cancer, gastrointestinal cancer, laryngeal cancer, leukemia, liver cancer, multiple myeloma, neuroblastoma, ovarian cancer, pancreatic cancer, prostatic cancer, retinoblastoma, skin cancer (e.g., melanoma and non-melanoma), stomach cancer, testicular cancer, thymus cancer, and thyroid cancer, as well as other carcinomas and sarcomas.

Other pathologic states are also contemplated in the context of the invention. For example, the pathologic state can be an inflammatory disease (e.g., arthritis), a neurodegenerative disease, a disease of an organ which is attributed to the presence of the increased or decreased level of a particular gene product(s), or any other pathologic state for which the site-specific integration and subsequent expression of a nucleic acid sequence encoding a therapeutic factor will treat or prevent a particular pathologic state.

Suitable methods, both invasive and noninvasive methods, of directly administering an expression construct (e.g., an expression construct composition) are available. Although more than one route can be used for administration, a particular route can provide a more immediate and more effective reaction than another route. The inventive method is not dependent on the mode of administering the expression construct composition to a mammal, preferably a human, to achieve the desired effect. As such, any route of administration is appropriate so long as the expression construct contacts and enters a cell within which integration is achievable. The composition can be appropriately formulated and administered in the form of a local injection, lotion, ointment, implant, or the like. The expression construct composition can be applied, for example, topically, intratumoraly, or peritumoraly. The expression construct composition can be administered through multiple applications and/or multiple routes to ensure sufficient exposure of cells to the expression construct composition.

The expression construct is preferably formulated into a composition prior to administration and is administered as soon as possible after it has been determined that an animal, such as a mammal, specifically a human, is at risk for a particular pathologic state (prophylactic treatment) or has begun to develop the pathologic state (therapeutic treatment). Treatment will depend, in part, upon the particular therapeutic factor expressed from the nucleic acid sequence, the route of administration, and the cause and extent, if any, of the pathologic state.

The expression construct can be administered using invasive procedures, such as, for instance, local injection (e.g., intratumoral injection). Intratumoral injections involve the administration of the expression construct composition directly into a tumor cell(s), which desirably selectively allow for expression construct replication. Pharmaceutically acceptable carriers for injectable compositions are well known to those of ordinary skill in the art (see Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986)).

The expression construct can be non-invasively administered to a mammal. For instance, if multiple surgeries have been performed, the mammal displays low tolerance to anesthetic, or other disorders exist, topical administration of the expression construct composition may be most appropriate. Topical formulations are well known to those of skill in the art. An expression construct composition also can be administered non-invasively using a needleless injection device, such as the Biojector 2000 Needle-Free Injection Management System® available from Bioject, Inc.

The expression construct is preferably present in or on a device that allows controlled or sustained release, such as a biocompatible polymeric matrix, meshwork, mechanical reservoir, or mechanical implant. Implants (see, e.g., U.S. Pat. Nos. 5,443,505, 4,853,224 and 4,997,652) and devices (see, e.g., U.S. Pat. Nos. 5,554,187, 4,863,457, 5,098,443 and 5,725,493), such as an implantable device, e.g., a mechanical reservoir or an implant or a device comprised of a polymeric composition, are particularly useful for the administration of the expression construct composition. The expression construct also can be administered in the form of a sustained-release formulation (see, e.g., U.S. Pat. No. 5,378,475) comprising, for example, gelatin, chondroitin sulfate, a polyphosphoester, such as bis-2-hydroxyethyl-terephthalate (BHET), or a polylactic-glycolic acid.

When administering the expression construct, the appropriate dosage and route of administration can be selected to minimize loss of the expression construct or inactivation of the expression construct due to a host's immune system. For example, for contacting cells in vivo, it can be advantageous to administer, to a mammal being treated, an immunosuppressive agent (e.g., cyclophosphamide or FK506) or monoclonal antibody that can block a T cell receptor, prior to performing the inventive method. Prior administration of an immunosuppressive agent or monoclonal antibody can serve to decrease the amount of expression construct cleared by the immune system of the mammal.

When practiced in vivo, any suitable organs or tissues or component cells can be targeted for expression construct delivery. Preferably, the organs/tissues/cells employed are of the circulatory system (i.e., heart, blood vessels or blood), respiratory system (i.e., nose, pharynx, larynx, trachea, bronchi, bronchioles, lungs), gastrointestinal system (i.e., mouth, pharynx, esophagus, stomach, intestines, salivary glands, pancreas, liver, gallbladder), urinary system (i.e., kidneys, ureters, urinary bladder, urethra), nervous system (i.e., brain and spinal cord, and special sense organs such as the eye) and integumentary system (i.e., skin). Even more preferably, the cells being targeted are selected from the group consisting of heart, blood vessel, lung, liver, gallbladder, urinary bladder, and eye cells.

The dose of expression construct administered to a mammal, particularly a human, in accordance with the invention should be in an amount sufficient to treat prophylactically or therapeutically a mammal for a pathologic state. Dosage will depend upon a variety of factors, including the age, species, the pathology in question, and condition or disease state. Dosage also depends on the nucleic acid sequences contained in the expression construct, as well as the amount of tissue about to be affected or actually affected by the disease. The size of the dose also will be determined by the route, timing, and frequency of administration as well as the existence, nature, and extent of any adverse side effects that might accompany the administration of a particular expression construct and the desired physiological effect. It will be appreciated by one of ordinary skill in the art that various conditions or disease states, in particular, chronic conditions or disease states, may require prolonged treatment involving multiple administrations.

Suitable doses and dosage regimens can be determined by conventional range-finding techniques known to those of ordinary skill in the art. When administering an expression construct, preferably about 10⁶particles to about 10¹² particles (e.g., naked DNA particles, plasmid particles, viral particles) are delivered to the diseased tissue. In other words, a composition of the expression construct can be administered that comprises an expression construct concentration of about 10⁶ particles/ml to about 10¹² particles/ml (including all integers within the range of about 10⁶ particles/ml to about 10¹² particles/ml), preferably about 10¹⁰ particles/ml to about 10¹² particles/ml. Typically, about 0.1 μl to about 100 μl of such an expression construct composition to each affected tissue. Of course, other routes of administration may require smaller or larger doses to achieve a therapeutic effect. Any necessary variations in dosages and routes of administration can be determined by the ordinarily skilled artisan using routine techniques known in the art.

In some embodiments, it is advantageous to administer two or more (i.e., multiple) doses of the expression construct. The invention provides for multiple applications of the expression construct in order to achieve sufficient integration, thereby prophylactically or therapeutically treating a particular disease state. For example, at least two applications of an expression construct can be administered to the same tissue. Preferably, the cell(s) is contacted with two applications or more of the expression construct via direct administration to the desired tissue within about 30 days or more. More preferably, two or more applications are administered to cells of the same tissue within about 90 days or more. However, three, four, five, six, or more doses can be administered in any time frame (e.g., 2, 7, 10, 14, 21, 28, 35, 42, 49, 56, 63, 70, 77, 85 or more days between doses) so long as the desired therapeutic effect is achieved. However, because the expression constructs of the invention are extremely efficient in integration, multiple applications are likely unnecessary.

The expression construct can be introduced ex vivo into cells, previously removed from the mammal, especially a human, and exposed to the expression construct, although this is less preferred. Such transduced autologous or homologous host cells, reintroduced into the mammal (e.g., human), will express directly the nucleic acid sequences contained therein in vivo following initiation of DNA replication. One ex vivo therapeutic option involves the encapsidation of infected cells into a biocompatible capsule, which can be implanted into a particular tissue. Such cells need not be isolated from the patient, but can instead be isolated from another individual and implanted into the patient.

The following examples further illustrate the invention but, of course, should not be construed as in any way limiting its scope.

### EXAMPLE 1

This example illustrates that a cis-acting element, other than AAV ITRs, functions as an integration efficiency element.

Two recombinant AAV (rAAV) plasmids were constructed from adeno-associated virus 2 (pRepGFP and pGFPCap). pRepGFP was constructed to contain, from 5′ to 3+, an AAV ITR, the Rep ORF operably linked to a nucleic acid sequence encoding an AAV IEE, a GFP transgene operably linked to a CMV promoter, and a second AAV ITR. pGFPCap was constructed to contain, from 5′ to 3′, an AAV ITR, a nucleic acid sequence encoding an AAV IEE, a GFP transgene operably linked to a CMV promoter, the second half of the wt AAV genome including the AAV Cap ORF, and a second AAV ITR. In addition, pTRUF2, which is a well-known rAAV plasmid, was utilized to analyze the integration efficiency of an expression construct which lacks a nucleic acid sequence encoding an AAV IEE. In that respect, pTRUF2 contains, from 5′ to 3′, an AAV ITR, a GFP transgene and a neomycin resistance gene operably linked to distinct promoters, and a second AAV ITR (see, e.g., Zolotukhin et al., J. Virol., 70:4646-4654 (1996)).

HeLa cells were co-transfected with pSub201 and either of pRepGFP, pGFPCap, or pTRUF2. pSub201 is a plasmid carrying the entire wtAAV genome. It is co-transfected with a corresponding rAAV plasmid to provide Rep in trans. Transfected cells were sorted by fluorescence-activated cell sorting (FACS) after 48 hours (Beckman-Coulter Altra cell sorter). The sorted cells were plated on a 96 well plate with 1 cell/well, and were allowed to grow for approximately 6 weeks. After this time, whole cell DNA was isolated from HeLa cells using a standard protocol (see, e.g., Miller et al., Nucleic Acids Res., 16:1215 (1988)). PCR and Southern Blots were performed to screen for GFP expression to determine if integration had occurred. The results of these experiments are summarized in Table 1.

TABLE 1

| Plasmid | Integration Efficiency Rate |
|---|---|
| pRepGFP | 27% |
| pGFPCap | 5% |
| pSub201 | 12% |
| pTRUF2 | <1% |

As indicated by the results set forth in Table 1, pRepGFP integrated at a rate of approximately 27%, substantially higher than the 12% integration efficiency observed with pSub201. Also, the integration efficiency of pGFPCap was 5%, which is lower than the 12% integration efficiency observed with pSub201 but higher than that observed with pTRUF2. These results demonstrate that the nucleic acid sequence encoding an AAV IEE (which was present in pRepGFP and pGFPCap, but absent in pTRUF2) enables efficient transgene integration.

### EXAMPLE 2

This example demonstrates that the deletion of AAV ITRs in a recombinant AAV plasmid does not influence the efficiency of Rep mediated site-specific integration.

Plasmids were constructed as described in Example 1 but with complete deletions of AAV ITRs. HeLa cells were co-transfected with pSub201 and either of pRepGFP(ITR−), pGFPCap(ITR−), or pTRUF2(ITR−). Transfected cells were sorted, plated, and grown as described in Example 1. Whole cell DNA was isolated from HeLa cells as described in Example 1. PCR and Southern Blots were performed to screen for GFP expression to determine if site-specific integration had occurred. The results are summarized in Table 2.

TABLE 2

| Plasmid | Integration Efficiency Rate |
|---|---|
| pRepGFP(ITR−) | 27% |
| pGFPCap(ITR−) | 5% |
| pSub201(ITR−) | 12% |
| pTRUF2(ITR−) | <1% |

As indicated by the results set forth in Table 2, expression constructs lacking AAV ITRs integrated into the host cell genome by Rep-mediated site-specific integration at a similar efficiency to their ITR-containing counterparts. These results demonstrate that AAV ITRs are not necessary for enabling site-specific integration, and, moreover, that a nucleic acid sequence encoding an AAV IEE is sufficient for directing site-specific integration of a transgene (e.g., in the absence of AAV ITRs).

### EXAMPLE 3

This example further demonstrates that AAV-ITR elements are not required for AAV site-specific integration.

HeLa cells were transfected with a pAAV/Ad plasmid (pRepCap(itr−)) (Samulski et al., J. Virol., 63, 3822-3828 (1989)) and a pCMV-GFB plasmid (Philpott et al., Proc. Natl. Acad. Sci., 99(19), 12381-12385 (2002)). pRepCap(itr−) contains wild-type AAV sequences with both flanking ITR elements deleted. pCMV-GFB expresses GFP and does not contain an AAV sequence. As a control, HeLa cells were transfected with a pSub201 plasmid (pRepCap(itr+)) (containing wild-type AAV sequences including both flanking ITR elements) and a pCMV-GFB plasmid.

Transfected cells were sorted by fluorescence-activated cell sorting (FACS) after 48 hours (Beckman-Coulter Altra cell sorter). The sorted cells were plated on a 96 well plate with 1 cell/well, and the cells were allowed to grow for approximately 6 weeks. After this time, genomic DNA was isolated from HeLa cells using a standard protocol (see, e.g., Miller et al., supra). PCR and Southern Blots were performed to screen for GFP expression to determine if integration had occurred and for the disruption of the AAVS1 genomic locus. The results are summarized in Table 3.

TABLE 3

| | | Percentage | Percentage of |
|---|---|---|---|
| | Number of | of clones | clones with |
| Plasmids | tested clones | with Rep | AAVS1 disruption |
| pRepCap(itr+) + pCMV- | 48 | 12 | 33 |
| GFB | | | |
| pRepCap(itr−) + pCMV- | 49 | 22 | 45 |
| GFB | | | |

As indicated above, the wild-type ITR-containing plasmid, pRepCap(itr+), had a Rep integration efficiency of 12%. AAVSI disruptions were found in a higher proportion of cell lines (33%) than those which retained the Rep DNA sequence.

In cells containing pRepCap(itr−) plasmid, Rep was found in 22% of the cell lines tested, and in most cases Rep was integrated into AAVS1. Furthermore, a large percentage (45%) of pRepCap(itr−) transfected cell lines had AAVSI genomic fragment disruptions.

From these results, it is apparent that plasmid constructs that lack the AAV ITR elements can serve as substrate DNA for rep-mediated site-specific integration into human chromosome 19 at the AAVSI site. Furthermore, it appears that higher levels of AAVS1 disruption than substrate DNA integrations are found in cell lines established from either ITR+or ITR− substrate plasmids.

This results of this experiment indicate that ITR elements are not required for site-specific integration into AAVS1, and that pRepCap(itr−) is an independent substrate for Rep-mediated site-specific integration into AAVS1.

### EXAMPLE 4

This example also demonstrates that AAV-ITR elements are not required for AAV site-specific integration.

It is commonly observed that when AAV integrates into HeLa AAVS1 sites, the resulting disruptions are variable in AAVS1 restriction fragment length and fragment band intensity. This phenomena may be explained by the influence of several factors on the character of AAVS1 integrants. For example, these factors may include the instability of the AAVS1 integration site (particularly in the presence of Rep), the aneuploidy of HeLa cells, and the imprecise alteration of the sequence of the AAVS1 site by the deletion-insertion mechanism of Rep-mediated AAVS1 integrations.

HeLa cells were cotransfected with a Rep-expressing plasmid to mediate the integration event and a GFP plasmid for FACS sorting transduced cells. Specifically, HeLa cells were transfected with the Rep-expressing plasmid, pRepCap(itr−) and pGFPCap (both described above). pGFPCap contains the first 7% of the AAV genome, followed by a GFP transgene and AAV Cap sequence.

Transfected cells were sorted by fluorescence-activated cell sorting (FACS) after 48 hours (Beckman-Coulter Altra cell sorter). The sorted cells were plated on a 96 well plate with 1 cell/well, and the cells were allowed to grow for approximately 6 weeks. After this time, genomic DNA was isolated from HeLa cells using a standard protocol (see, e.g., Miller et al., supra). PCR and Southern Blots were performed to screen for GFP expression to determine if integration had occurred and for the disruption of the AAVS1 genomic locus.

Approximately 6% of the 78 tested cell lines contained integrated pGFPCap DNA. When the cell lines were screened for genomic alterations of the AAVS1 site of chromosome 19, 54% of the clones were found to have AAVS1 site disruptions. As observed in Example 3, it appears that AAVS1 disruptions occurred at much higher frequencies than pGFPCap integration events. This observation may be explained by one or more of the following: Rep-mediated disruption of AAVS1 sites can occur in the absence of integration; integration events are occurring at AAVS1 sites but the integrated DNA is unstable, or DNA elements other than pGFPCap are integrating at the AAVS1 site.

14% of the cell lines contained Rep DNA that in most cases was site-specifically integrated. If the ITRs were required for AAV site-specific integration, such a large percentage of integrants would not have been observed. Since it was possible that a recombination event between the pGFPCap and pRepCap(itr−) plasmids to form wild-type AAV comprising ITR elements (the plasmids share significant homology at both the 5′ and 3′ ends of their respective AAV regions) was responsible for the large number of Rep integrants, PCR analysis of genomic DNA was performed using one primer complementary to the pRepCap(itr−) Rep sequence and one primer complementary to the pGFPCap ITR sequence. A PCR product of about 700 bp would indicate a wild-type recombination event, whereas neither of the two plasmids separately would serve as a substrate for the PCR primer pair. A PCR product was not obtained from any of the pGFPCap-pRepCap(itr−) co-transfected cell lines, which suggested that recombination between pRepCap(itr−) and pGFPCap to form a wild-type AAV plasmid was unlikely to have accounted for the Rep integrants. This result supports the finding that pRepCap(itr−) is an independent substrate for Rep-mediated site-specific integration into AAVS1 (i.e., ITR elements are not required for Rep-mediated site specific integration into AAVS1).

### EXAMPLE 5

This example demonstrates that ITR elements influence the boundaries of integration substrates.

As described in Example 3, genomic DNA was isolated from the pRepCap(itr−) and the pRepCap(itr+) transfected clones (each of which was co-transfected with pCMV-GFB). The DNA was digested with Eco R1 and separated on 1% agarose gels. The resulting DNA was transferred to nylon membranes and hybridized to a ³²P-labeled probe of the pRepCap(itr−) plasmid backbone sequence. The results of this analysis are summarized in Table 4.

TABLE 4

| | | Percentage of | Percentage |
|---|---|---|---|
| | Number of | clones with | site- |
| Plasmids | tested clones | plasmid backbone | specificity |
| pRepCap(itr+) + pCMV- | 45 | 23 | 90 |
| GFB | | | |
| pRepCap(itr−) + pCMV- | 49 | 24 | 91 |
| GFB | | | |

The Southern blot of genomic DNA from pRepCap(itr−)-transfected cells probed with plasmid backbone showed 24% of the cell lines contained plasmid backbone. Additionally, 91% of the Rep-positive clones were positive for the plasmid backbone. Based on these results, it appears that the entire plasmid sequence was the substrate for AAVS1 integration in cell lines derived from a pRepCap(itr−) transduction of HeLa cells.

The Southern blot of genomic DNA from pRepCap(itr+)-transfected cells probed with plasmid backbone resulted in a more complex pattern of AAVS1 integration. 27% of cell lines tested had at least part of the pRepCap(itr+) sequence integrated. Rep was site-specifically integrated into 12% of the cell lines (as discussed in Example 3). 23% of the cell lines were shown to contain plasmid integrants.

The three types of integrants observed following co-transfection of pRepCap(itr+) and pCMV-GFB may be explained as follows. The terminal hairpin structure of the AAV ITR serves as the viral origin of replication. In a double-stranded plasmid substrate, such as in pRepCap(itr+), duplex cruciform structures can be generated at each ITR, with each cruciform being a substrate for Rep binding and nicking. If nicking at an ITR cruciform occurs, it may result in defining the segment of pRepCap(itr+) element that is able to undergo site-specific integration. If this occurs, three distinct integration substrates may result: RepCap flanked by ITR elements, plasmid backbone flanked by ITR elements, or both RepCap and plasmid backbone flanked by ITR elements. In contrast, pRepCap(itr−) lacks the complexity and function of the ITR elements, and all integrants of pRepCap(itr−) should include the entire pRepCap(itr−) DNA sequence.

The overall integration efficiency of ITR-containing or ITR-deleted constructs (27% and 24%, respectively) was similar. Therefore, the AAV ITRs made a minimal contribution toward integration efficiency. However, the presence of the AAV ITR elements can act to generate integration boundaries when using a plasmid integration substrate. Presumably, Rep binding to the Rep-binding elements (RBEs) and nicking at the terminal resolution site (trs) of the ITR generates potential 5′ and 3′ boundaries of the integration substrate. In a wild-type viral infection, boundary definition is naturally present due to the ITR hairpin structure present at the ends of the linear, single-stranded viral genome.

### EXAMPLE 6

This example demonstrates that the only cis-element that is necessary and sufficient for site-specific integration at AAVS1 is present in the sequence comprising the AAV integration efficiency element (IEE) and p5 promoter region. The p5 promoter region of AAV overlaps with the IEE.

The plasmid pAd-p5CAT containing the p5 promoter region and AAVIEE upstream of a chloramphenicol acetyl transferase (CAT) reporter gene was constructed (Philpott et al., Proc. Natl. Acad. Sci., supra). Promoter activity was confirmed with this construct in transient transfection assays. In HeLa cells, the p5 promoter was able to mediate expression of CAT levels comparable to the CMV promoter and was vulnerable to trans repression by Rep.

To determine the function of the construct in an integration assay, HeLa cells were co-transfected with pAd-p5CAT and the Rep-expressing plasmid pT7-Rep (Philpott et al., J. Virol., 76, 5411-5421 (2002)). The pT7-Rep plasmid expressed GFP, which allowed for FACS sorting 24 hours post-transfection. Cell lines were established as previously described, and each cell line was harvested for genomic DNA. Genomic DNA was digested with Eco R1, and a Southern blot analysis was performed probing for the presence of CAT or for the presence of AAVS1 disruptions. The results are summarized in Table 5.

TABLE 5

| | | Percentage of | Percentage | |
|---|---|---|---|---|
| | Number | clones with | with | Percentage |
| | of tested | plasmid | AAVS1 | site- |
| Plasmids | clones | backbone | disruption | specificity |
| pT7-Rep + pAd- | 45 | 22 | 29 | 100 |
| p5CAT | | | | |
| pAd-p5CAT | 49 | 0 | 0 | 0 |

Out of 45 cell lines tested, 29% contained AAVS1 disruptions, and CAT gene integrations were found in 22% of cell lines. In all cases, pAd-p5CAT DNA co-migrated with the AAVS1 probe (i.e. 100% site specificity to the AAVSl site).

As a negative control, HeLa cells were transfected with pAd-p5CAT alone. In the absence of a Rep-expressing plasmid, the CAT transgene was unable to integrate, and this result confirms that the targeted integration of a substrate is Rep-dependent.

The results of this experiment indicate that the sequence comprising the p5 promoter region and IEE is the only AAV element required in cis to mediate site-specific integration of a substrate DNA through Rep-dependent integration into AAVS1.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An expression construct comprising a nucleic acid sequence encoding an adeno-associated virus integration efficiency element (AAV IEE), wherein the expression construct is substantially devoid of AAV inverted terminal repeats (AAV ITRs), and wherein the expression construct site-specifically integrates into a host cell chromosome when provided to the host cell in conjunction with an AAV Rep protein.

2. The expression construct of claim 1, wherein the Rep protein comprises a long form of Rep.

3. The expression construct of claim 1, wherein the expression construct is a viral vector.

4. The expression construct of claim 3, wherein the viral vector is an adenoviral vector.

5. The expression construct of claim 4, wherein the adenoviral vector is deficient in one or more replication-essential gene functions.

6. The expression construct of claim 1, wherein the expression construct further comprises a nucleic acid sequence of interest.

7. The expression construct of claim 6, wherein the nucleic acid sequence of interest encodes a protein.

8. The expression construct of claim 7, wherein the protein is a therapeutic factor.

9. The expression construct of claim 8, wherein the nucleic acid sequence of interest encoding a therapeutic factor is positioned downstream of the AAV IEE.

10. The expression construct of claim 8, wherein the therapeutic factor is useful to prophylactically or therapeutically treat a mammal for a pathologic state.

11. A composition comprising the expression construct of claim 1 and a carrier.

12. A host cell comprising the expression construct of claim 1.

13. The host cell of claim 12, wherein the host cell is propagated to produce a stable cell line.

14. A method of integrating a nucleic acid sequence of interest into a host cell chromosome comprising:
(a) providing to a host cell an expression construct comprising a nucleic acid sequence encoding an adeno-associated virus integration efficiency element (AAV IEE) and a nucleic acid sequence of interest, wherein the expression construct is substantially devoid of AAV inverted terminal repeats (AAV ITRs), and
(b) providing to the host cell a nucleic acid sequence encoding an AAV Rep protein,
such that the nucleic acid sequence encoding the AAV Rep protein is expressed, thereby resulting in the site-specific integration of the nucleic acid sequence of interest into a chromosome contained in the host cell.

15. The method of claim 14, wherein the AAV Rep protein is provided to the host cell in trans.

16. The method of claim 14, wherein the AAV Rep protein is provided to the host cell in the expression construct of (a).

17. The method of claim 14, wherein the nucleic acid sequence of interest encodes a protein.

18. The method of claim 17, wherein the protein is a therapeutic factor.

19. The method of claim 18, wherein the therapeutic factor is useful to prophylactically or therapeutically treat a mammal for a pathologic state.

20. The method of claim 14, wherein the expression construct is a viral vector.

21. The method of claim 20, wherein the viral vector is an adenoviral vector.

22. The method of claim 21, wherein the adenoviral vector is deficient in one or more replication-essential gene functions.

23. The method of claim 22, wherein the host cell is propagated to create a stable cell line.

24. A method of prophylactically or therapeutically treating a mammal for a pathologic state comprising:
(a) administering to a mammal an expression construct comprising a nucleic acid sequence encoding an adeno-associated virus integration efficiency element (AAV IEE) and a nucleic acid sequence encoding a therapeutic factor, wherein the expression construct is substantially devoid of AAV inverted terminal repeats (AAV ITRs), and
(b) administering to the mammal a nucleic acid sequence encoding an AAV Rep protein,
such that the nucleic acid sequence encoding the AAV Rep protein is expressed, thereby resulting in the site-specific integration of the nucleic acid sequence encoding a therapeutic factor into a chromosome of a host cell of the mammal, expression of the nucleic acid sequence encoding a therapeutic factor, and subsequent production of the therapeutic factor to prophylactically or therapeutically treat the mammal for the pathologic state.

25. The method of claim 24, wherein the AAV Rep protein is provided to the host cell in trans.

26. The method of claim 24, wherein the AAV Rep protein is provided to the host cell in the expression construct of (a).

27. The method of claim 24, wherein the pathologic state is cancer.

28. The method of claim 24, wherein the expression construct is a viral vector.

29. The method of claim 28, wherein the viral vector is an adenoviral vector.

30. The method of claim 29, wherein the adenoviral vector is deficient in one or more replication-essential gene functions.
